(19) United States
(12) Patent Application Publication
(10) Pub. No.: US 2007/0027184 A1
(43) Pub. Date: Feb. 1, 2007

(54) Multicyclic sulfonamide compounds as inhibitors of histone deacetylase for the treatment of disease

(75) Inventors: James W. Malecha, San Diego, CA (US); Stewart A. Noble, San Diego, CA (US); Brandon M. Wiley, Philadelphia, PA (US); Timothy Z. Hoffman, San Diego, CA (US); Celine Bonnefous, San Diego, CA (US); Michael Sertic, Euclid, OH (US); Paul L. Wash, San Diego, CA (US); Nicholas D. Smith, San Diego, CA (US); Christian A. Hassig, Mira Mesa, CA (US); Shawn A. Scranton, San Diego, CA (US); Joseph E. Payne, Oceanside, CA (US); Jeffery Hager, San Diego, CA (US)

Correspondence Address: INTERNATIONAL PATENT GROUP;ATTN: MS LAVERN HALL, P.O. BOX 38129, ST. LOUIS, MO, 63138, US

(73) Assignee: KALYPSYS, INC.

(21) Appl. No.: 11/496,784

(22) Filed: Jul. 27, 2006

Related U.S. Application Data
(60) Provisional application No. 60/704,091, filed on Jul. 29, 2005.
(60) Provisional application No. 60/780,129, filed on Mar. 7, 2006.

(51) Int. Cl. A61K 31/47; C07D 215/12; C07D 319/14; A61K 31/36

(52) U.S. Cl. 514/311; 514/465; 514/452; 546/176; 549/362; 549/454

(57) ABSTRACT

Disclosed herein are sulfonamide compounds of Formula VII as described herein. 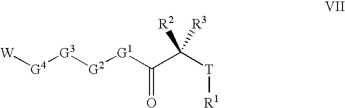 Methods and compositions are disclosed for treating disease states including, but not limited to cancers, autoimmune diseases, tissue damage, central nervous system disorders, neurodegenerative disorders, fibrosis, bone disorders, polyglutamine-repeat disorders, anemias, thalassemias, inflammatory conditions, cardiovascular conditions, and disorders in which angiogenesis play a role in pathogenesis, using the compounds of the invention. In addition, methods of modulating the activity of histone deacetylase (HDAC) are also disclosed.

This application claims the benefit of priority of U.S. provisional application No. 60/704,091, filed Jul. 29, 2005 and provisional application No. 60/780,129 filed Mar. 7, 2006, the disclosure of which is hereby incorporated by reference as if written herein in its entirety.

### FIELD OF THE INVENTION

The present invention is directed to multicyclic sulfonamide compounds as inhibitors of histone deacetylase (HDAC). These compounds are useful in treating disease states including cancers, autoimmune diseases, tissue damage, central nervous system disorders, neurodegenerative disorders, fibrosis, bone disorders, polyglutamine-repeat disorders, anemias, thalassemias, inflammatory conditions, cardiovascular conditions, and disorders in which angiogenesis plays a role in pathogenesis.

### BACKGROUND OF THE INVENTION

Histone proteins organize DNA into nucleosomes, which are regular repeating structures of chromatin. The acetylation status of histones alters chromatin structure, which, in turn, is involved in gene expression. Two classes of enzymes can affect the acetylation of histones—histone acetyltransferases (HATs) and histone deacetylases (HDACs). A number of HDAC inhibitors have been characterized. One of the potent inhibitors of HDAC is (SAHA), a hydroxamic acid-based compound. It is also known as vorinostat or ZOLINZA™, which is currently in clinical trials. (“Merck Announces Pivotal Phase IIb Study Results of the Company's Investigational HDAC Inhibitor ZOLINZA™ and Glaxo's Cancer Vaccine Shows Response,” M2 Presswire, 5 Jun. 2006.) The Food and Drug Administration (FDA) has also accepted the New Drug Application (NDA) for ZOLINZA™ for the treatment of advanced cutaneous T-cell-lymphoma (CTCL) in June 2006. (WHITEHOUSE STATION, N.J., “ZOLINZA™, Merck's Investigational Medicine for Advanced Cutaneous T-Cell Lymphoma (CTCL), to Receive Priority Review from U.S. Food and Drug Administration,” Business Wire, 7 Jun. 2006.)

### SUMMARY OF THE INVENTION

Disclosed herein are sulfonamide compounds of Formula VII and related Formula III, as described herein, including their pharmaceutically acceptable salts, esters, and prodrugs. Compounds of Formula VII have the following structure

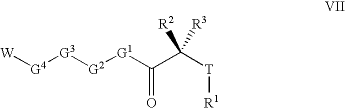

or a therapeutically acceptable salt, ester, or prodrug, thereof, wherein:

G¹ is selected from the group consisting of a bond, alkenyl, alkoxy, alkoxyalkyl, alkyl, alkylamino, alkylcarbonyl, alkylcarbonylalkyl, alkylcarbonylamino, alkylcarbonylaminoalkyl, alkynyl, amino, aminoalkyl, carbonylalkyl, and carbonylaminoalkyl;

G² is selected from the group consisting of optionally substuteted monocyclic heteroaryl, and optionally substuteted polycyclic heteroaryl;

G³ is selected from the group consisting of —X¹SO₂N(R⁷)— and —X¹N(R⁷)SO₂—;

X¹ is selected from the group consisting of a bond or an alkyl of length C₁ to C₃, any carbon atom of which may be optionally substituted;

R⁷ is selected from the group consisting of hydrogen, alkenyl, and alkyl, or alternatively, R⁷ may be joined to G² to form a heterocyclo or heteroaryl ring;

G⁴ is selected from the group consisting of bicyclic aryl, bicyclic heteroaryl, cycloalkyl-fused monocyclic aryl, cycloalkyl-fused monocyclic heteroaryl, heterocycloalkyl-fused monocyclic aryl, and heterocycloalkyl-fused monocyclic heteroaryl, wherein each may be optionally substituted;

T is selected from the group consisting of O and S;

W is selected from the group consisting of null and —U¹X²U²;

U¹ is selected from the group consisting of a bond, heterocycloalkyl, —NR¹⁰ —, —O—, —S—, —C(O)N(R¹⁰)—, —N(R¹⁰)C(O)—, —S(O)₂N(R¹⁰), and —N(R¹⁰)S(O)—;

R¹⁰ is selected from the group consisting of hydrogen, alkenyl, and alkyl;

U² is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted;

X² is selected from the group consisting of a bond or an alkyl of length C₁ to C₇, any carbon atom of which may be optionally substituted;

R² and R³ are independently selected from the group consisting of hydrogen, methyl, and ethyl;

R¹ is selected from the group consisting of hydrogen, —P(O)(OR¹⁴)OR¹⁵, cyano, optionally substuteted acyl, aroyl, aryl, alkyl, heteroaryl, heterocycloalkyl, carboxy, carboxyalkyl, optionally substituted alkylthio, optionally substituted arylthio, and a group of structural Formula II

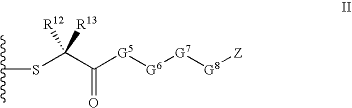

R¹⁴ and R¹⁵ are independently selected from the group consisting of hydrogen, alkyl, aryl, and heteroaryl;

R¹² and R¹³ are independently selected from the group consisting of hydrogen, methyl, and ethyl;

G⁵ are independently selected from the group consisting of a bond, alkenyl, alkoxy, alkoxyalkyl, alkyl, alkylamino, alkylcarbonyl, alkylcarbonylalkyl, alkylcarbonylamino, alkylcarbonylaminoalkyl, alkynyl, amino, aminoalkyl, carbonylalkyl, and carbonylaminoalkyl;

G⁶ are independently selected from the group consisting of optionally substuteted monocyclic heteroaryl, and optionally substuteted polycyclic heteroaryl;

G⁷ is selected from the group consisting of —X³SO₂N(R⁸)— and —X³N(R⁸)SO₂—;

X³ is selected from the group consisting of a bond or an alkyl of length C₁ to C₃, any carbon atom of which may be optionally substituted;

R⁸ is selected from the group consisting of hydrogen, alkenyl, and alkyl, or alternatively, R⁸ may be joined to G⁵ to form a heterocyclo or heteroaryl ring;

G⁸ is selected from the group consisting of bicyclic aryl, bicyclic heteroaryl, cycloalkyl-fused monocyclic aryl, cycloalkyl-fused monocyclic heteroaryl, heterocycloalkyl-fused monocyclic aryl, and heterocycloalkyl-fused monocyclic heteroaryl, wherein each may be optionally substituted;

Z is selected from the group consisting of null and —U³X⁴U⁴;

U³ is selected from the group consisting of a bond, heterocycloalkyl, —NR¹¹—, —O—, —S—, —C(O)N(R¹¹)—, —N(R¹¹)C(O)—, —S(O)₂N(R¹¹)—, and —N(R¹¹)S(O)—;

R¹¹ is selected from the group consisting of hydrogen, alkenyl, and alkyl;

U⁴ is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted; and

X⁴ is selected from the group consisting of a bond or an alkyl of length C₁ to C₇, any carbon atom of which may be optionally substituted.

Compounds according to the present invention possess useful HDAC inhibitory activity, and may be used in the treatment or prophylaxis of a disease or condition in which HDAC plays an active role. Thus, in broad aspect, the present invention also provides pharmaceutical compositions comprising one or more compounds of the present invention together with a pharmaceutically acceptable carrier, as well as methods of making and using the compounds and compositions. In certain embodiments, the present invention provides methods for inhibiting the catalytic activity and cellular function of histone deacetylase (HDAC). In other embodiments, the present invention provides methods for treating an HDAC-mediated disorder in a patient in need of such treatment comprising administering to said patient a therapeutically effective amount of a compound or composition according to the present invention. The present invention also contemplates the use of compounds disclosed herein for use in the manufacture of a medicament for the treatment of a disease or condition ameliorated by the inhibition of HDAC.

### DETAILED DESCRIPTION OF THE INVENTION

A preferred family of compounds consists of compounds of Formula I wherein 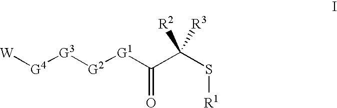
G¹ is a bond.

In certain embodiments, T is S.

In further embodiments, R¹ is hydrogen.

In yet further embodiments, R¹ is acyl.

In certain embodiments, both R² are hydrogen.

In certain embodiments, G³ is selected from the group consisting of —X¹SO₂N(R⁷)— and —X¹N(R⁷)SO₂—, and X¹ is a bond.

In other embodiments, G² is an optionally substituted 6-membered heteroaryl.

In certain embodiments, G⁴ is an optionally substituted napthyl.

In further embodiments, G⁴ is an optionally substituted bicyclic heteroaryl.

In yet further embodiments, G⁴ is an optionally substituted cycloalkyl-fused monocyclic aryl.

In yet further embodiments, G⁴ is a heterocycloalkyl-fused monocyclic aryl, cycloalkyl-fused monocyclic heteroaryl, or heterocycloalkyl-fused monocyclic heteroaryl, wherein each may be optionally substituted.

In some embodiments, G² is an optionally substituted polycyclic heteroaryl.

In certain embodiments, G⁴ is an optionally substituted napthyl.

In further embodiments, G⁴ is an optionally substituted bicyclic heteroaryl.

In yet further embodiments, G⁴ is an optionally substituted cycloalkyl-fused monocyclic aryl.

In yet further embodiments, G⁴ is a heterocycloalkyl-fused monocyclic aryl, cycloalkyl-fused monocyclic heteroaryl, or heterocycloalkyl-fused monocyclic heteroaryl, wherein each may be optionally substituted.

A more preferred embodiment of the present invention is a compound of the Formula III:

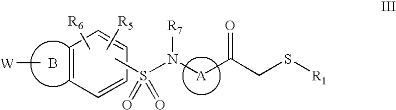

or a therapeutically acceptable salt, ester, or prodrug, thereof, wherein:

A is a six-membered heteroaryl ring or polycyclic heteroaryl;

B is a saturated or unsaturated hydrocarbon chain or a saturated or unsaturated heteroatom-comprising hydrocarbon chain having from 3 to 5 atoms, forming a five- to seven-membered ring;

W is selected from the group consisting of null and —U¹X²U²;

U¹ is selected from the group consisting of a bond, heterocycloalkyl, —NR¹⁰—, —O—, —S—, —C(O)N(R¹⁰)—, —N(R¹⁰)C(O)—, —S(O)₂N(R¹⁰), and —N(R¹⁰)S(O)—;

U² is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted;

X² is selected from the group consisting of a bond or an alkyl of length C₁ to C₇, any carbon atom of which may be optionally substituted;

R¹ is selected from the group consisting of hydrogen, —P(O)(OR¹⁴)OR¹⁵, cyano, acyl, aryl, alkyl, heteroaryl, heterocycloalkyl and Z, wherein Z has the structural Formula IV

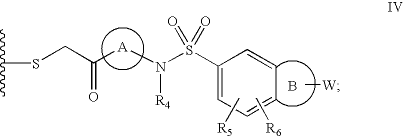

R⁴ is selected from the group consisting of hydrogen, alkenyl, and alkyl;

R¹⁴ and R¹⁵ are each independently selected from the group consisting of hydrogen, alkyl, aryl, and heteroaryl;

R⁵ and R⁶ are each independently selected from the group consisting of hydrogen, alkyl, heteroalkyl, alkenyl, alkoxy, alkoxyalkyl, cyano, halo, haloalkoxy, haloalkyl, hydroxyl, amino and nitro; and

R¹⁰ is selected from the group consisting of hydrogen, alkenyl, and alkyl.

In certain embodiments, R¹ is hydrogen or acyl.

In some embodiments, A is a six-membered heteroaryl ring.

In further embodiments, B comprises a chain having four atoms and forming a six-membered ring.

In further embodiments, two of the said four atoms of B are heteroatoms selected from the group consisting of N, O, and S.

In yet further embodiments, B has the structural Formula V

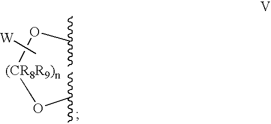

R⁸ and R⁹ are each independently selected from the group consisting of hydrogen, lower alkyl, lower alkenyl, and lower alkynyl;

n is an integer from 1 to 3; W is null.

In certain embodiments, n is 2 and both R⁸ and R⁹ are hydrogen.

In other embodiments, R⁷ is hydrogen.

In certain embodiments, A is a pyridyl ring.

In other embodiments, B has the structural Formula VI

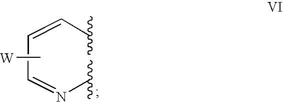

In further embodiments, U¹ is selected from the group consisting of a bond, heterocycloalkyl, —NR¹⁰—, —O—; and

X² is selected from the group consisting of a bond or an alkyl of length C₁ to C₄, any carbon atom of which may be optionally substituted.

In some embodiments, R⁷ is hydrogen.

In other embodiments, A is a pyridyl ring.

In certain embodiments, R¹ is selected from the group consisting of optionally substituted alkylthio and optionally substituted arylthio.

In further embodiments, said alkylthio is substituted with one or more of an amino substituent and a carboxylic acid substituent.

In yet further embodiments, said alkylthio is substituted with both an amino substituent and a carboxylic acid substituent.

In another aspect, the invention relates to a compounds selected from the group consisting of Examples 1-24, or a pharmaceutically acceptable salt, ester, amide, or prodrug.

Yet another aspect of the invention is Example 20.

In some aspects of the present invention are compounds containing at least one thiol in a protected form, which can be released to provide a SH group prior to or simultaneous to use. Thiol moieties are known to be unstable in the presence of air and are oxidized to the corresponding disulfide. Protected thiol groups are those that can be converted under mild conditions into gree thiol groups without other undesired side reactions taking place. Suitable thiol protecting groups include but are not limited to trityl (Trt), allyloxycarbonyl (Alloc), 1-(4,4-dimethyl-2,6-dioxocyclohex-1-ylidene)ethyl (Dde), acetamidomethyl (Acm), t-butyl (tBu), or the like. Preferred thiol protecting groups include lower alkanoyl, e.g. acetyl. Free thiol, disulfides, and protected thiols are understood to be within the scope of this invention.

In accordance with yet another aspect of the invention, the present invention provides methods and compositions for treating certain diseases.

In some aspects of the invention, the disease is a hyperproliferative condition of the human or animal body.

In further embodiments, said hyperproliferative condition is selected from the group consisting of hematologic and nonhematologic cancers. In yet further embodiments, said hematologic cancer is selected from the group consisting of multiple myeloma, leukemias, and lymphomas. In yet further embodiments, said leukemia is selected from the group consisting of acute and chronic leukemias. In yet further embodiments, said acute leukemia is selected from the group consisting of acute lymphocytic leukemia (ALL) and acute nonlymphocytic leukemia (ANLL). In yet further embodiments, said chronic leukemia is selected from the group consisting of chronic lymphocytic leukemia (CLL) and chronic myelogenous leukemia (CML). In further embodiments, said lymphoma is selected from the group consisting of Hodgkin's lymphoma and non-Hodgkin's lymphoma. In further embodiments, said hematologic cancer is multiple myeloma. In other embodiments, said hematologic cancer is of low, intermediate, or high grade. In other embodiments, said nonhematologic cancer is selected from the group consisting of: brain cancer, cancers of the head and neck, lung cancer, breast cancer, cancers of the reproductive system, cancers of the digestive system, pancreatic cancer, and cancers of the urinary system. In further embodiments, said cancer of the digestive system is a cancer of the upper digestive tract or colorectal cancer. In further embodiments, said cancer of the urinary system is bladder cancer or renal cell carcinoma. In further embodiments, said cancer of the reproductive system is prostate cancer.

Additional types of cancers which may be treated using the compounds and methods described herein include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. In certain embodiments, these cancers may be selected from the group consisting of: cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic.

Yet other types of cancers which may be treated using the compounds and methods described herein include: adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, and Wilm's tumor.

In some aspects of the invention, the disease to be treated by the methods of the present invention may be a hematologic disorder. In certain embodiments, said hematologic disorder is selected from the group consisting of sickle cell anemia, myelodysplastic disorders (MDS), and myeloproliferative disorders. In further embodiments, said myeloproliferative disorder is selected from the group consisting of polycythemia vera, myelofibrosis and essential thrombocythemia.

In some aspects of the invention, the disease to be treated by the methods of the present invention may be a neurological disorder. In further embodiments, said neurological disorder is selected from the group consisting of epilepsy, neuropathic pain, depression and bipolar disorders.

In some aspects of the invention, the disease to be treated by the methods of the present invention may be a cardiovascular condition. In certain embodiments, said cardiovascular condition is selected from the group consisting of cardiac hypertrophy, idiopathic cardiomyopathies, and heart failure.

In some aspects of the invention, the disease to be treated by the methods of the present invention may be an autoimmune disease. In certain embodiments, said autoimmune disease is selected from the group consisting of systemic lupus erythromatosus (SLE), multiple sclerosis (MS), and systemic lupus nephritis.

In some aspects of the invention, the disease to be treated by the methods of the present invention may be a dermatologic disorder. In certain embodiments, said dermatologic disorder is selected from the group consisting of psoriasis, melanoma, basal cell carcinoma, squamous cell carcinoma, and other non-epithelial skin cancers.

In some aspects of the invention, the disease to be treated by the methods of the present invention may be an ophthalmologic disorder. In certain embodiments, said ophthalmologic disorder is selected from the group consisting of dry eye, closed angle glaucoma and wide angle glaucoma.

In some aspects of the invention, the disease to be treated by the methods of the present invention may be a polyglutamine-repeat disorders. In some embodiments, the polyglutamine-repeat disorder is selected from the group consisting of Huntington's disease, Spinocerebellar ataxia 1 (SCA 1), Machado-Joseph disease (MJD)/Spinocerebella ataxia 3 (SCA 3), Kennedy disease/Spinal and bulbar muscular atrophy (SBMA) and Dentatorubral pallidolusyian atrophy (DRPLA).

In some aspects of the invention, the disease to be treated by the methods of the present invention may be an inflammatory condition. In some embodiments, the inflammatory condition is selected from the group consisting of Rheumatoid Arthritis (RA), Inflammatory Bowel Disease (IBD), ulcerative colitis and psoriasis.

In further aspects of the invention, the the disease to be treated by the methods of the present invention may be a disorder related to bone remodeling or resorption. In certain aspects, said condition may be selected from the group consisting of osteoporosis and formation of osteoclasts.

In another aspect are compounds or compositions comprising compounds capable of inhibiting the catalytic or cellular activity of histone deacetylase (HDAC).

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. More preferred alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-diethylamino and the like.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfanyl,” as used herein, alone or in combination, refers to an alkyl group attached to the parent molecular moiety through a sulfanyl group. Examples of alkylsulfanyl groups include methylsulfanyl, ethylsulfanyl, butylsulfinyl and hexylsulfanyl. Alkylsulfanyl groups may be optionally substituted as described herein. Examples of substituted alkylsulfanyl groups include aminoalkylsulfanyl and carboxyalkylsulfanyl.

The term “alkylsulfinyl,” as used herein, alone or in combination, refers to an alkyl group attached to the parent molecular moiety through a sulfinyl group. Examples of alkylsulfinyl groups include methylsulfinyl, ethylsulfinyl, butylsulfinyl and hexylsulfinyl.

The term “alkylsulfonyl,” as used herein, alone or in combination, refers to an alkyl group attached to the parent molecular moiety through a sulfonyl group. Examples of alkylsulfinyl groups include methanesulfonyl, ethanesulfonyl, tert-butanesulfonyl, and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, C≡C—). Examples of alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like.

The term “amido,” as used herein, alone or in combination, refers to an amino group as described below attached to the parent molecular moiety through a carbonyl group, or an acyl group attached to the parent moiety through an amino group. The term “C-amido” as used herein, alone or in combination, refers to a —C(═O)—NR² group with R as defined herein. The term “N-amido” as used herein, alone or in combination, refers to a RC(═O)NH— group, with R as defined herein.

The term “amino,” as used herein, alone or in combination, refers to —NRR′, wherein R and R′ are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted with one, two, three, four, or five substituents independently selected from the group consisting of alkenyl, alkoxy, alkoxyalkyl, alkyl, cyano, halo, haloalkoxy, haloalkyl, hydroxy, hydroxy-alkyl, nitro, and oxo.

The term “aminoalkyl,” as used herein, alone or in combination, refers to an amino group attached to the parent molecular moiety through an alkyl group. Examples include aminomethyl, aminoethyl and aminobutyl.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an —C(—CN)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl —O—C(O) in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy -2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylamino” as used herein, alone or in combination, refers to an aryl group attached to the parent moiety through an amino group, such as methylamino, N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “benzo” and “benz,” as used herein, alone or in combination, refer to the divalent radical C₆H₄═ derived from benzene. Examples include benzothiophene and benzimidazole.

The term “bicyclic” as used herein is intended to refer to two saturated or unsaturated (i.e., aromatic) cyclic rings in which two atoms are common to two adjoining rings, e.g., the rings are “fused rings”. Aryl groups can be fused to another aryl groups or cycloalkyl groups. For examples, “cycloalkyl-fused mono heteroaryl” means a cycloalkyl ring fused with a monocylic heteroaryl ring.

The term “O-carbamyl” as used herein, alone or in combination, refers to a —OC(O)NR, group—with R as defined herein.

The terms “carboxy” or “carboxyl”, whether used alone or with other terms, such as “carboxyalkyl”, denotes —CO₂H.

The term “N-carbamyl” as used herein, alone or in combination, refers to a ROC(O)NH— group, with R as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “carboxyalkyl,” as used herein, refers to —C(O)OH or —C(O)OR attached to the parent moiety through an alkyl group.

The term “cyano,” as used herein, alone or in combination, refers to —CN.

The term “cycloalkyl,” as used herein, alone or in combination, refers to an aliphatic cyclic alkyl moeity wherein the ring is either completely saturated, partially unsaturated, or fully unsaturated, wherein if there is unsaturation, the conjugation of the pi-electrons in the ring do not give rise to aromaticity. The term “cycloalkyl” may refer to a monocyclic or polycyclic group. Cycloalkyl groups may be fused or linked to other cyclic alkyl moeities. A cycloalkyl group may be optionally substituted. Preferred cycloalkyl groups include groups having from three to twelve ring atoms, more preferably from 5 to 10 ring atoms. The term “carbocyclic cycloalkyl” refers to a monocyclic or polycyclic cycloalkyl group which contains only carbon and hydrogen. The term “heterocycloalkyl” refers to a monocyclic or polycyclic cycloalkyl group wherein at least one ring backbone contains at least one atom which is different from carbon.

The term “disulfide”, as used herein, refers to a disulfide ion or two sulfur atoms bonded together. A disulfide ion is an anion formed by two sulfur atoms. Disulfides of the invention are either asymmetric or symmetric. Preferred disulfides are symmetric and in a preferred embodiment, compounds of Structures I are provided by the invention wherein T=S, and R¹=Z, wherein G⁵=G² and G⁶=G⁴ so as to form a symmetric disulfide dimmer.

The term “ester,” as used herein, alone or in combination, refers to a carboxyl group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (—CFH—), difluoromethylene (—CF₂—), chloromethylene (—CHCl—) and the like. Examples of such haloalkyl radicals include chloromethyl, 1-bromoethyl, fluoromethyl, difluoromethyl, trifluoromethyl, 1,1,1-trifluoroethyl, perfluorodecyl and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic groups containing an oxygen atom, for example, pyranyl, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.]etc.; unsaturated condensed heterocyclic groups containing 1 to 2 oxygen atoms and 1 to 3 nitrogen atoms [e.g. benzoxazolyl, benzoxadiazolyl, etc.]; unsaturated 3 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.]and isothiazolyl; unsaturated condensed heterocyclic groups containing 1 to 2 sulfur atoms and 1 to 3 nitrogen atoms [e.g., benzothiazolyl, benzothiadiazolyl, etc.] and the like. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of such fused polycyclic groups include fused bicyclic groups such as benzofuryl, benzothienyl, thienopyridine, furopyridine, pyrrolopyridine and the like.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylalkoxy,” as used herein, alone or in combination, refers to a heteroaryl group attached to the parent molecular moiety through an alkoxy group.

The term “heteroalkyl” or “heteroarylalkyl,” as used herein, alone or in combination, refers to a heteroaryl group attached to the parent molecular moiety through an alkyl group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylsulfonyl,” as used herein, alone or in combination, refers to a heteroaryl group attached to the parent molecular moiety through a sulfonyl group.

The term “heterocycloalkyl,” as used herein, alone or in combination, refers to a saturated, partially unsaturated, or fully unsaturated monocyclic, bicyclic, or tricyclic heterocyclic radical containing at least one heteroatom as ring member, wherein each said heteroatom may be independently selected from the group consisting of nitrogen, oxygen and sulfur. Heterocycloalkyl groups may be fused with one or more aryl, heteroaryl, cycloalkyl, or heterocycloalkyl groups. Heterocycloalkyl groups may be linked with one or more aryl, heteroaryl, cycloalkyl, or heterocycloalkyl groups. Examples of heterocycloalkyl (non-aromatic heterocyclic groups) are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl.

The term “heterocycloalkenyl,” as used herein, alone or in combination, refers to a heterocycle group attached to the parent molecular moiety through an alkenyl group.

The term “heterocycloalkoxy,” as used herein, alone or in combination, refers to a heterocycle group attached to the parent molecular group through an oxygen atom.

The term “heterocycloalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl” as used herein, alone or in combination, refers to a linear or branched alkyl group having one to about ten carbon atoms any one of which may be substituted with one or more hydroxyl radicals. Examples of such radicals include hydroxymethyl, hydroxyethyl, hydroxypropyl, hydroxybutyl and hydroxyhexyl.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′SR— group, where R and R′ are as defined herein.

The term “mercaptomercaptyl” as used herein, alone or in combination, refers to a RSR′S— group, where R is as defined herein.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “polycyclic” as used herein is intended to refer to two or more saturated or unsaturated (i.e., aromatic) cyclic rings in which two atoms are common to two adjoining rings, e.g., the rings are “fused rings”. Polycyclic aryl groups may be fused. Polycyclic aryl groups can be fused to aryl groups or cycloalkyl groups. For examples, “cycloalkyl-fused mono- or polycyclic heteroaryl” means a cycloalkyl ring fused with either a monocylic heteroaryl ring or a polycyclic heteroaryl ring.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S and —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NH— group with R as defined herein.

The term “S-sulfonamido” refers to a —S(═O)₂NR², group, with R as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thioether,” as used herein, alone or in combination, refers to a thio group bridging two moieties linked at carbon atoms.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NH— group, with R as defined herein.

The term “O-thiocarbamyl” refers to a OC(S)NR, group with R as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

The term “optionally substituted” means the anteceding group may be substituted or unsubstituted. When substituted, the substituents of an “optionally substituted” group may include, without limitation, one or more substituents independently selected from the following groups or designated subsets thereof, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, NHCH₃, N(CH₃)₂, SH, SCH₃, C(O)CH₃, CO₂CH₃, CO₂H, C(O)NH₂, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH2CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended.

When a group is defined to be “null,” what is meant is that said group is absent.

A group can be attached to the corresponding atom of attachment in either order. For example, —NHC(O)—, can be attached through either the nitrogen atom or the carbon atom to the core structure.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term R or the term R′, appearing by itself and without a number designation, unless otherwise defined, refers to an optionally substituted moiety selected from the group consisting of alkyl, cycloalkyl, heteroalkyl, aryl, heteroaryl and heterocycloalkyl. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

When a substituent, such as R⁷, shown by way of example in two alternatives below: 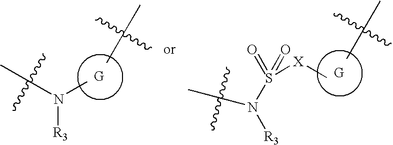
is said to be joined to a ring, such as G, to form another ring, then the following is meant:

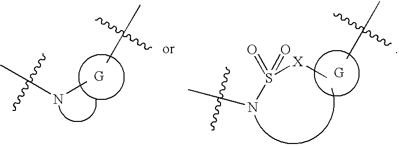

In the above example, R⁷ joined to G to from another ring, resulting in a fused ring system. Unless otherwise specified, such polycyclic ring fusion can occur with a carbon atom or a heteroatom present in G.

The term “bond” refers to a covalent linkage between two atoms, or two moieties when the atoms joined by the bond are considered to be part of larger substructure. A bond may be single, double, or triple unless otherwise specified.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The terms “therapy” or “treating” as used herein refer to (1) reducing the rate of progress of a disease, or, in case of cancer reducing the size of the tumor; (2) inhibiting to some extent further progress of the disease, which in case of cancer may mean slowing to some extent, or preferably stopping, tumor metastasis or tumor growth; and/or, (3) relieving to some extent (or, preferably, eliminating) one or more symptoms associated with the disease. Thus, the term “therapeutically effective amount” as used herein refers to that amount of the compound being administered which will provide therapy or affect treatment.

In some aspects of the invention, the compounds of the present invention are also anti-tumor compounds and/or inhibit the growth of a tumor, i.e., they are tumor-growth-inhibiting compounds. The terms “anti-tumor” and “tumor-growth-inhibiting,” when modifying the term “compound,” and the terms “inhibiting” and “reducing”, when modifying the terms “compound” and/or “tumor,” mean that the presence of the subject compound is correlated with at least the slowing of the rate of growth of the tumor. More preferably, the terms “anti-tumor,” “tumor-growth-inhibiting,” “inhibiting,” and “reducing” refer to a correlation between the presence of the subject compound and at least the temporary cessation of tumor growth. The terms “anti-tumor,” “tumor-growth-inhibiting,” “inhibiting,” and “reducing” also refer to, a correlation between the presence of the subject compound and at least the temporary reduction in the mass of the tumor.

The term “function” refers to the cellular role of HDAC. The term “catalytic activity”, in the context of the invention, defines the rate at which HDAC deacetylates a substrate. Catalytic activity can be measured, for example, by determining the amount of a substrate converted to a product as a function of time. Deacetylation of a substrate occurs at the active-site of HDAC. The active-site is normally a cavity in which the substrate binds to HDAC and is deacetylated.

The term “substrate” as used herein refers to a molecule deacetylated by HDAC. The substrate is preferably a peptide and more preferably a protein. In some embodiments, the protein is a histone, whereas in other embodiments, the protein is not a histone.

The term “inhibit” refers to decreasing the cellular function of HDAC. It is understood that compounds of the present invention may inhibit the cellular function of HDAC by various direct or indirect mechanisms, in particular by direct or indirect inhibition of the catalytic activity of HDAC. The term “activates” refers to increasing the cellular function of HDAC.

The term “activates” refers to increasing the cellular function of HDAC. The term “inhibit” refers to decreasing the cellular function of HDAC. HDAC function is preferably the interaction with a natural binding partner and most preferably catalytic activity.

The term “modulates” refers to altering the function of HDAC by increasing or decreasing the probability that a complex forms between HDAC and a natural binding partner. A modulator may increase the probability that such a complex forms between HDAC and the natural binding partner, or may increase or decrease the probability that a complex forms between HDAC and the natural binding partner depending on the concentration of the compound exposed to HDAC, or may decrease the probability that a complex forms between HDAC and the natural binding partner. A modulator may activate the catalytic activity of HDAC, or may activate or inhibit the catalytic activity of HDAC depending on the concentration of the compound exposed to HDAC, or may inhibit the catalytic activity of HDAC.

The term “complex” refers to an assembly of at least two molecules bound to one another. The term “natural binding partner” refers to polypeptides that bind to HDAC in cells. A change in the interaction between HDAC and a natural binding partner can manifest itself as an increased or decreased probability that the interaction forms, or an increased or decreased concentration of HDAC/natural binding partner complex.

The term “contacting” as used herein refers to mixing a solution comprising a compound of the invention with a liquid medium bathing the cells of the methods. The solution comprising the compound may also comprise another component, such as dimethylsulfoxide (DMSO), which facilitates the uptake of the compound or compounds into the cells of the methods. The solution comprising the compound of the invention may be added to the medium bathing the cells by utilizing a delivery apparatus, such as a pipet-based device or syringe-based device.

The term “monitoring” refers to observing the effect of adding the compound to the cells of the method. The effect can be manifested in a change in cell phenotype, cell proliferation, HDAC catalytic activity, substrate protein acetylation levels, gene expression changes, or in the interaction between HDAC and a natural binding partner.

The term “effect” describes a change or an absence of a change in cell phenotype or cell proliferation. “Effect” can also describe a change or an absence of a change in the catalytic activity of HDAC. “Effect” can also describe a change or an absence of a change in an interaction between HDAC and a natural binding partner.

The term “cell phenotype” refers to the outward appearance of a cell or tissue or the function of the cell or tissue. Examples of cell phenotype are cell size (reduction or enlargement), cell proliferation (increased or decreased numbers of cells), cell differentiation (a change or absence of a change in cell shape), cell survival, apoptosis (cell death), or the utilization of a metabolic nutrient (e.g., glucose uptake). Changes or the absence of changes in cell phenotype are readily measured by techniques known in the art.

“HDAC inhibitor” is used herein to refer to a compound that exhibits an IC₅₀ with respect to HDAC activity of no more than about 100 .mu.M and more typically not more than about 50 μM, as measured in the in vitro HDAC-inhibition assay, cellular histone hyperacetylation assay, and differential cytotoxicity assay described generally hereinbelow. “IC₅₀” is that concentration of inhibitor which reduces the activity of an enzyme (e.g., HDAC) to half-maximal level. Representative compounds of the present invention have been discovered to exhibit inhibitory activity against HDAC. Compounds of the present invention preferably exhibit an IC₅₀ with respect to HDAC of no more than about 10 μM, more preferably, no more than about 5 μM, even more preferably not more than about 1 μM, and most preferably, not more than about 200 nM, as measured in the HDAC assays described herein.

The phrase “therapeutically effective” is intended to qualify the amount of active ingredients used in the treatment of atherosclerosis. This amount will achieve the goal of reducing or eliminating the hyperlipidemic condition.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Yet another example of a prodrug are protected thiol compounds. Thiols bearing hydrolyzable protecting groups can unmask protected SH groups prior to or simultaneous to use. As shown below, the moiety —C(O)—R_{E} of a thioester may be hydrolyzed to yield a thiol and a pharmaceutically acceptable acid HO—C(O)—R_{E}.

A “pharmaceutically active metabolite” is intended to mean a pharmacologically active product produced through metabolism in the body of a specified compound or salt thereof. Metabolites of a compound may be identified using routine techniques known in the art and their activities determined using tests such as those decribed herein.

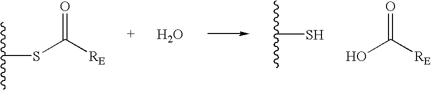

The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “thiol protecting group” refers to thiols bearing hydrolyzable protecting groups that can unmask protected SH groups prior to or simultaneous to use. Preferred thiol protecting groups include but are not limited to thiol esters which release pharmaceutically acceptable acids along with an active thiol moiety. Such pharmaceutically acceptable acids are generally nontoxic and do not abbrogate the biological activity of the active thiol moiety. Examples of pharmaceutically acceptable acids include, but are not limited to: N,N-diethylglycine; 4-ethylpiperazinoacetic acid; ethyl 2-methoxy-2-phenylacetic acid; N,N-dimethylglycine; (nitrophenoxysulfonyl)benzoic acid; acetic acid; maleic acid; fumaric acid; benzoic acid; tartraric acid; natural amino acids (like glutamate, aspartate, cyclic amino acids such proline); D-amino acids; butyric acid; fatty acids like palmitic acid, stearic acid, oleate; pipecolic acid; phosphonic acid; phosphoric acid; pivalate (trimethylacetic acid); succinic acid; cinnamic acid; anthranilic acid; salicylic acid; lactic acid; and pyruvic acids.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use.

The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question.

The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In one embodiment, pharmaceutical preparations of compound(s) or active ingredient(s) of the present invention may be formulated by Latitude Pharmaceuticals Inc. located in 9865 Mesa Rim Road, STE 201, San Diego, Calif. 92121 using their trade secret and proprietary formulation named “F101”. The composition of said formulation F101 is known to contain triglyceride, soy lecithin, vitamin E and PEG400.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

Further, the compounds of the invention may be administered on a daily basis or on a schedule containing days where dosing does not take place. In certain embodiments, dosing may take place every other day. In other embodiments, dosing may take place for five consecutive days of a week, then be followed by two non-dosing days. The choice of dosing schedule will depend on many factors, including, for example, the formulation chosen, route of administration, and concurrent pharmacotherapies, and may vary on a patient-to-patient basis. It is considered within the capacity of one skilled in the art to select a schedule that will maximize the therapeutic benefit and minimize any potential side effects in a patient.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with another chemotherapeutic agent such as aromatase inhibitors, antiestrogen, anti-androgen, or a gonadorelin agonists, topoisomerase 1 and 2 inhibitors, microtubule active agents, alkylating agents, antimeoplastic antimetabolite, or platin containing compound, lipid or protein kinase targeting agents, protein or lipid phosphatase targeting agents, anti-angiogentic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, and aminopeptidase inhibitors.

In some aspects of the invention, the chemotherapeutic agents that are useful for the treatment of Multiple Myeloma include, but are not limited to, alkylating agents (eg, melphalan), anthracyclines (eg. doxorubicin), corticosteroids (eg. dexamethasome), IMiDs (eg. Thalidomide, lenalidomide), protease inhibitors (eg. bortezomib, NP10052), IGF-1 inhibitors, CD40 antibody, Smac mimetics (eg. telomestatin), FGF3 modulator (eg. CHIR258), mTOR inhibitor (Rad 001), HDAC inhibitors (eg. SAHA, Tubacin), IKK inhibitors, P38MAPK inhibitors, HSP90 inhibitor (eg 17-AAG), and akt inhibitor (eg. Perifosine).

Further, the preferred chemotherapeutic agents used in combination with the compounds of the present invention, but without limitation, is selected from melphalan, doxorubicin (including lyophilized), dexamethasone, prednisone, thalidomide, lenalidomide, bortezomib, and NP10052.

In any case, the multiple chemotherapeutic agents (at least one of which is a compound of the present invention) may be administered in any order or even simultaneously. If simultaneously, the multiple chemotherapeutic agents may be provided in a single, unified form, or in multiple forms (by way of example only, either as a single pill or as two separate pills). One of the chemotherapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating HDAC-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of HDAC-mediated disorders.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

Molecular embodiments of the present invention can be synthesized using standard synthetic techniques known to those of skill in the art. Compounds of the present invention can be synthesized using the general synthetic procedures set forth in Schemes I-XI. 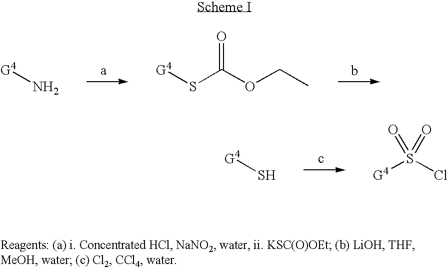 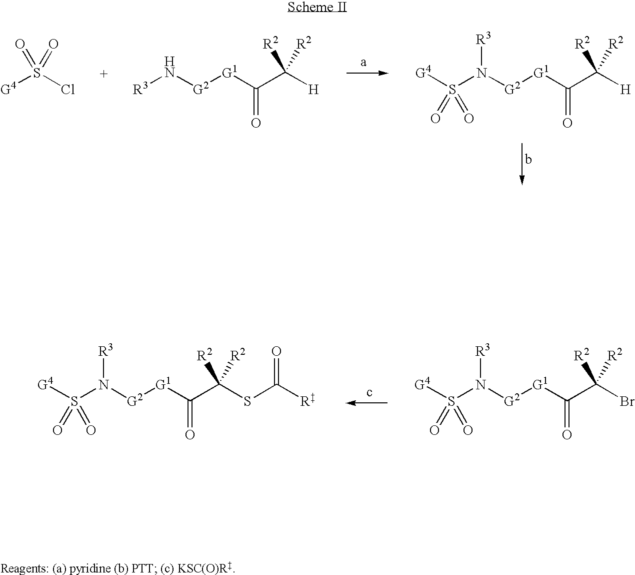 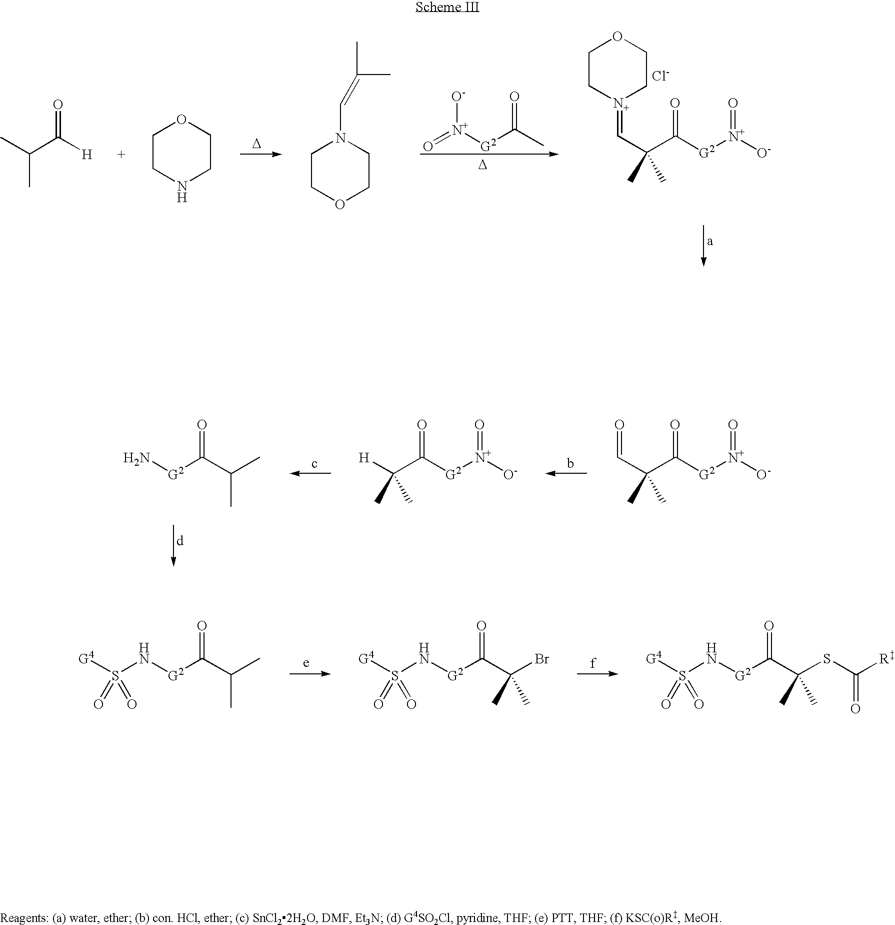 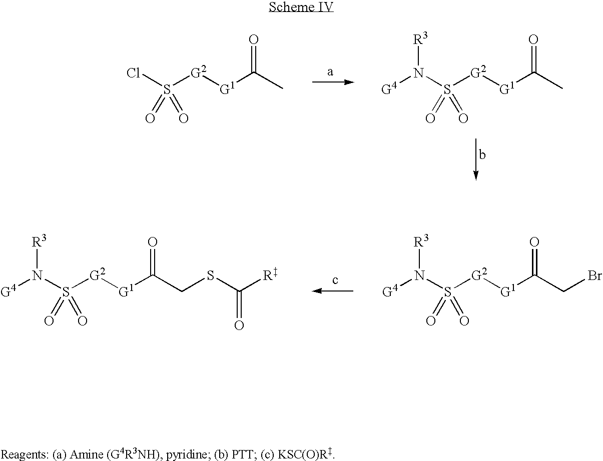 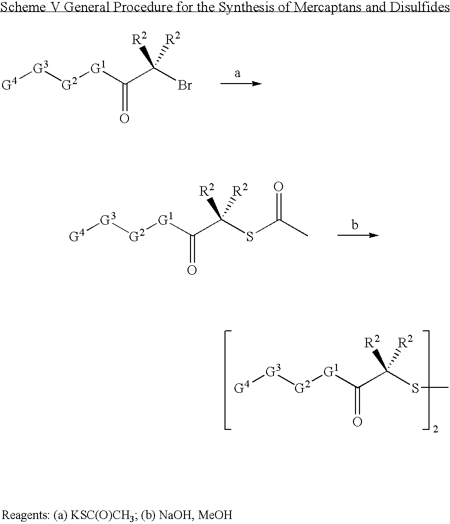 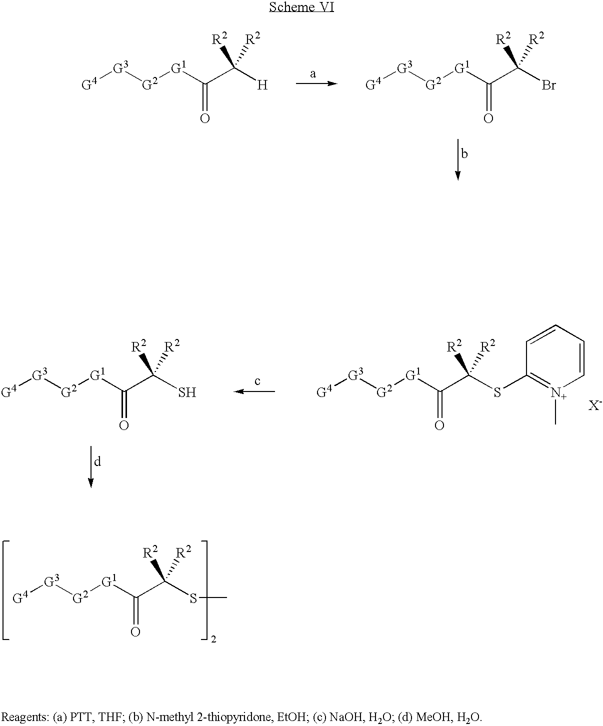 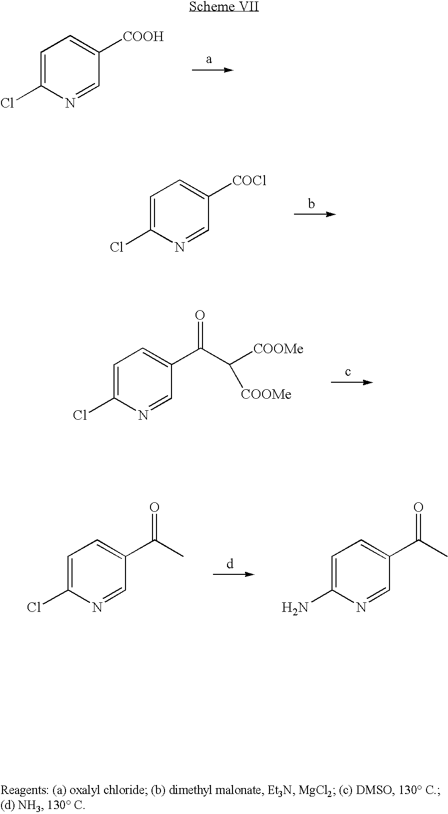 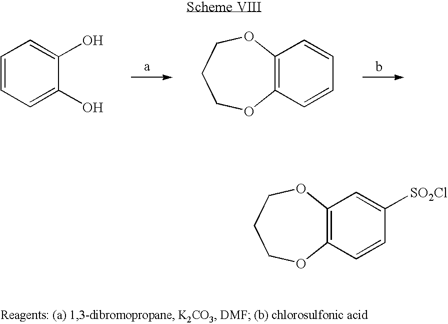 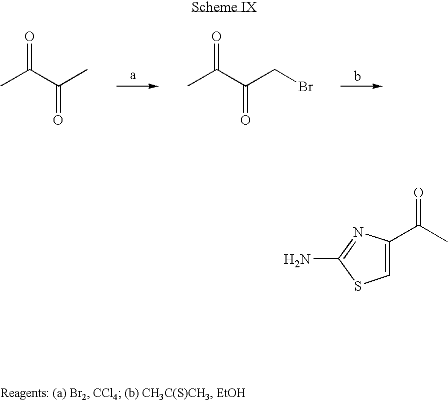 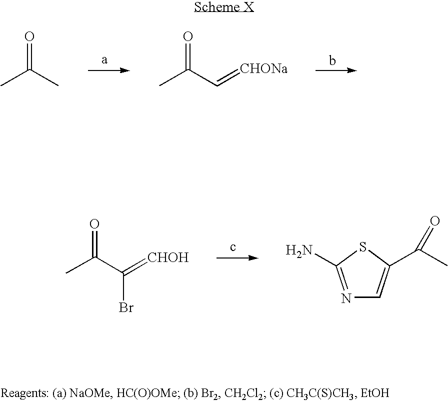 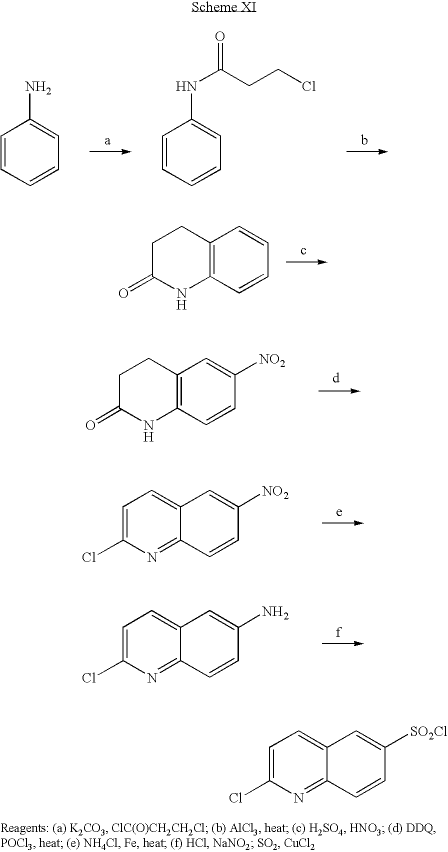
The invention is further illustrated by the following examples. All compound names below were generated by either ChemDraw 10.0 or ChemDraw 8.0.

### EXAMPLES

The examples below are non-limiting and are merely representative of various aspects of the invention.

### Example 1

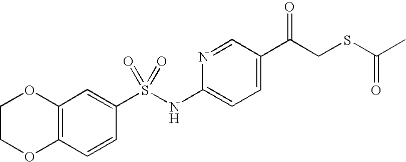

### Thioacetic acid S-{2-[6-(2,3-dihydro-benzol[1,4]dioxine-6-sulfonylamino)-pyridin-3-yl]-2-oxo-ethyl)}ester

Step 1 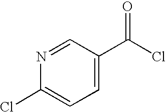
6-Chloronicotinoyl chloride: A mixture of 6-chloronicotinic acid (27.0 g, 172 mmol) and oxalyl dichloride (70 mL) was heated at 63° C. for 20 h. The mixture was cooled to room temperature and concentrated under reduced pressure to give the desired product, 31.6 g (98%), as a light yellow solid.
Step 2 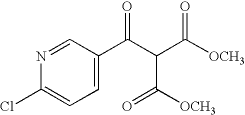
Dimethyl 2-(6-chloronicotinoyl)malonate: To a solution of magnesium chloride (29.1 g, 306 mmol) in toluene (400 mL) was added dimethyl malonate (69.6 g, 527 mmol) and triethylamine (106 g, 1.05 mol). The reaction mixture was stirred at room temperature for 1 h followed by the addition of 6-chloronicotinoyl chloride (77.0 g, 438 mmol) in toluene (150 mL). The reaction mixture was stirred at room temperature for 3.5 h and then poured into H₂O/ice (200 mL). The aqueous mixture was extracted from EtOAc (4×150 mL). The combined organic solution was washed with brine, dried and concentrated under reduced pressure to afford the desired product, 119.2 g (92%), as a brown solid.
Step 3 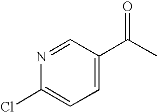
1-(6-Chloropyridin-3-yl)ethanone: A solution of dimethyl 2-(6-chloronicotinoyl)malonate (89.8 g, 331 mmol) in DMSO (445 mL) and water (11 mL) was heated at 130° C. for 2.5 hours. The reaction mixture was cooled and poured into H₂O/ice (300 mL). The aqueous mixture was extracted from EtOAc (4×150 mL). The combined organic solution was washed with brine, dried and concentrated under reduced pressure. The residue was recrystallized from 60% ethanol-water to give the desired compound, 165 g (32%), as a yellow solid.
Step 4

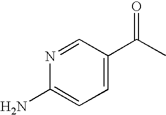

1-(6-Aminopyridin-3-yl)ethanone: Into a 1 L high pressure clave, was placed a solution of 1-(6-chloropyridin-3-yl)ethanone (40 g, 257.10 mmol) in saturated ammonium (750 ml). The reaction mixture was stirred at 130° C. for 10 h. The mixture was cooled and concentrated under reduced pressure. The residue was purified by silica gel chromatography (50:1 CH₂Cl₂/MeOH) to give the desired compound, 33 g (89%), as a yellow solid.
Step 5 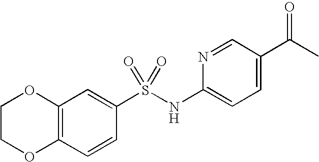
2,3-Dihydro-benzo[1,4]dioxine-6-sulfonic acid (5-acetyl-pyridin-2-yl)-amide: To a solution of 1-(6-amino-pyridin-3-yl)-ethanone (8.6 g, 63.2 mmol) in pyridine (48 mL) was added 2,3-dihydro-benzo[1,4]dioxine-6-sulfonyl chloride (13.5 g, 57.5 mmol). The reaction mixture was heated to 50° C. for 2 h. The mixture was cooled and poured into H₂O/ice (200 mL). The resulting precipitate was collected by filtration. The solid was washed with water and methanol then dried to give the desired product, 16.4 g, (86%) as a tan solid. LC-MS (ES+): 335 [MH]⁺ m/e.
Step 6 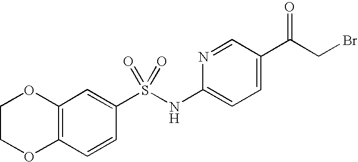
2,3-Dihydro-benzo[1,4]dioxine-6-sulfonic acid [5-(2-bromo-acetyl)-pyridin-2-yl]-amide: To 2,3-dihydro-benzo[1,4]dioxine-6-sulfonic acid (5-acetyl-pyridin-2-yl)-amide (5.78 g, 17.3 mmol) in DMF (25 mL) was added 32% HBr in acetic acid (5 mL, 26 mmol) over 20 min keeping the temperature below 25° C. To the reaction was then added phenyltrimethylammonium tribromide (PTT) (6.5 g, 17.3 mmol) and the mixture was stirred for 9.5 h. The mixture was poured into H₂O/ice (100 mL) and the resulting precipitate was collected by filtration. The solid was washed with water and methanol then recrystallized from acetone/water to give the desired compound, 5.8 g (81%). LC-MS (ES+): 412, 414 m/e.
Step 7 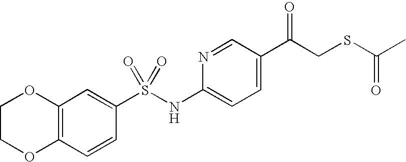
Thioacetic acid S-{2-[6-(2,3-dihydro-benzo[1,4]dioxine-6-sulfonylamino)-pyridin-3-yl]-2-oxo-ethyl)}ester: To a solution of 2,3-dihydro-benzo[1,4]dioxine-6-sulfonic acid [5-(2-bromo-acetyl)-pyridin-2-yl]-amide (2.12 g, 5.14 mmol) in methanol (20 mL) was added potassium thioacetate (646 mg, 5.66 mmol). The mixture was heated to 55° C. for 1 h. Volatiles were concentrated in vacuo to afford a tan residue which was taken up into DMSO and purified by HPLC-MS yielding the desired compound as a white solid (0.98 g, 47%). ¹H-NMR (400 MHz, DMSO-d6): δ 11.17 (s, 1H), 8.43 (d, 1H), 7.91 (d, 1H), 7.71 (dd, 1H), 7.34 (m, 2H), 7.04 (d, 1H), 4.53 (s, 2H), 4.29 (q, 4H), 2.35 (s, 3H); LC-MS (ES+): 409 [MH]⁺ m/e.

### Example 2

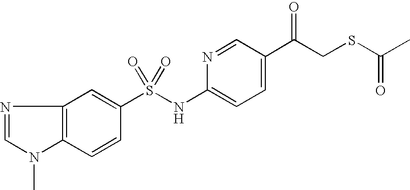

### Thioacetic acid S-{2-[6-(1-methyl-1H-benzoimidazole-5-sulfonylamino)-pyridin-3-yl]-2-oxo-ethyl)}ester

Step 1 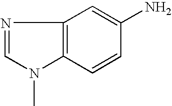
To a solution of 1-Methyl-5-nitro-1H-benzoimidazole (3.4 g, 19.2 mmol) in toluene (75 mL) was added Raney Nickel (1 g). The mixture was stirred at 40° C. under H₂ for 4 h. The reaction mixture was filtered though celite and concentrated in vacuo to give an orange solid. The crude solid was purified by silica gel chromatography (40-65% EtOAc/Hexanes) to give the desired amine as an orange solid (2.14 g, 76%). ¹H-NMR (400 MHz, CDCl₃): δ 7.77 (s, 1H), 7.22 (d, 1H), 6.93 (s, 1H), 6.88 (dd, 1H), 4.01 (s, 3H), 3.60 (s, 2H).
Step 2 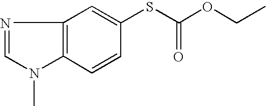
To the product from Step 1 (2.31 g, 15.7 mmol) in water (30 mL) at 0° C. was added conc. HCl (3.14 mL, 37.9 mmol). Sodium nitrite (1.16 g, 16.8 mmol) in water (5 mL) was added portionwise to the stirring solution of amine over a period of 30 minutes. The reaction was stirred at 0° C. for an additional 25 minutes and then added portionwise to a stirring 40-45° C. solution of potassium ethyl xanthate (2.93 g, 18.3 mmol) in water (10 mL). The reaction was stirred at 45° C. for an additional 30 minutes and then cooled to room temperature. The mixture was extracted with Et₂O (2×75 mL). The organic extracts were concentrated in vacuo to give a yellow oil. The crude oil was purified by silica gel chromatography to give the desired xanthate ester as a yellow oil (1.68 g, 42%). ¹H-NMR (400 MHz, CDCl₃): δ 8.02 (s, 1H), 7.90 (s, 1H), 7.45 (m, 2H), 4.62 (q, 2H), 4.11 (s, 3H), 1.33 (t, 3H).
Step 3 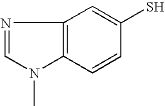
To a solution of the product from Step 2 (1.38 g, 5.47 mmol) in THF (21 mL) and MeOH (7 mL) was added lithium hydroxide (0.52 g, 21.9 mmol). Water was added (8 mL) and the mixture was heated to 60° C. for 3.5 hours. The mixture was diluted with water (300 mL) and washed with CH₂Cl₂ (2×100 mL). The aqueous solution was acidified to pH ˜1 with conc. HCl and extracted with CH₂Cl₂ (3×100 mL). The organic extracts were combined, dried over MgSO₄, and concentrated in vacuo to give the desired thiol as a white solid (0.76 g, 4.6 mmol, 85%). ¹H-NMR (400 MHz, CDCl₃): δ 7.91 (s, 1H), 7.73 (s, 1H), 7.37 (dd, 1H), 7.31 (d, 1H), 4.08 (s, 3H), 2.26 (s, 1H).
Step 4 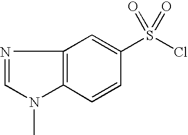
To a solution of the product from Step 3 (0.76 g, 4.6 mmol) in carbon tetrachloride (30 mL) was added water (5 mL) and the mixture was cooled to 0° C. Chlorine gas was bubbled through the mixture for 50 minutes. The mixture was diluted with CH₂Cl₂ (100 mL) and water (100 mL). The organic layer was separated, dried over magnesium sulfate, and concentrated in vacuo to leave the desired sufonyl chloride as a white solid (0.91 g, 85%). ¹H-NMR (400 MHz, CDCl₃): δ 8.53 (s, 1H), 8.22 (s, 1H), 8.01 (dd, 1H), 7.58 (d, 1H), 4.15 (s, 3H).
Steps 5-7 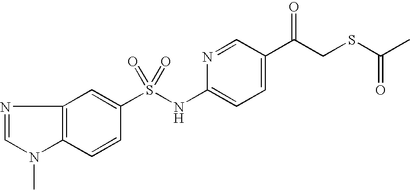
The compound was prepared according to the procedure described in Example 1 using the product of Step 4 as the starting material. ¹H-NMR (400 MHz, DMSO-d₆): δ 11.70 (s, 1H), 8.73 (s, 1H), 8.47 (s, 1H), 8.28 (s, 1H), 8.14 (d, 1H), 7.87 (d, 1H), 7.80 (d, 1H), 7.21 (s, 1H), 4.39 (s, 2H), 4.07 (s, 3H), 2.34 (s, 3H). LC-MS (ES+): 405 [MH]⁺ m/e.

### Example 3

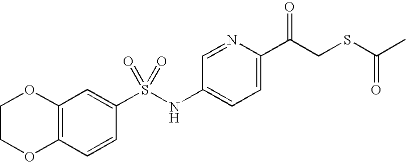 Thioacetic acid S-{2-[5-(2,3-dihydro-benzo[1,4]dioxine-6-sulfonylamino)-pyridin-2-yl]-2-oxo-ethyl}ester: The compound was prepared according to the procedure described in Example 1 using 1-(5-amino-pyridin-2-yl)-ethanone. ¹H-NMR (400 MHz, DMSO-d₆): 11.17 (s, 1H), 8.43 (d, 1H), 7.91 (d, 1H), 7.71 (dd, 1H), 7.34 (m, 2H), 7.04 (d, 1H), 4.53 (s, 2H), 4.29 (q, 4H), 2.35 (s, 3H). LC-MS (ES+): 409 [MH]⁺ m/e.

### Example 4

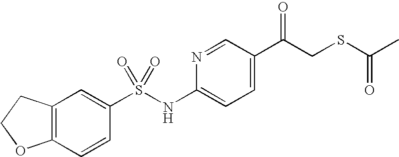 Thioacetic acid S-{2-[6-(2,3-dihydro-benzofuran-5-sulfonylamino)-pyridin-3-yl]-2-oxo-ethyl}ester: The compound was prepared according to the procedure described in Example 1 using 2,3-dihydro-benzofuran-5-sulfonyl chloride. ¹H-NMR: (400 MHz, CDCl₃) δ 9.01(s, 1H), 8.21(d, 1H), 7.76(d, 1H), 7.74(s, 1H), 7.41(d, 1H), 6.81(d, 1H), 4.65(t, 2H), 4.24(s, 2H), 3.24(t, 2H), 2.40(s, 3H). MS: (392.05)

### Example 5

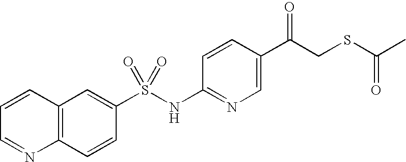

Thioacetic acid S-{2-oxo-2-[4-(quinoline-6-sulfonylamino)-phenyl]-ethyl}ester: The compound was prepared according to the procedure described in Example 1 using quinoline-6-sulfonyl chloride. ¹H-NMR (400 MHz, DMSO-d₆): δ 9.1 (m, 1H), 8.70 (m, 3H), 8.15 (m, 3H), 7.68 (q, 1H), 7.3 (s, 1H), 4.38 (s, 2H), 2.34 (s, 3H). LC-MS (ES+): 401 [MH]⁺ m/e

### Example 6

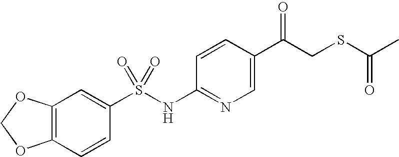 Thioacetic acid S-{2-[6-(benzo[1,3]dioxole-5-sulfonylamino)-pyridin-3-yl]-2-oxo-ethyl}ester: The compound was prepared according to the procedure described in Example 1 using benzo[1,3]dioxole-5-sulfonyl chloride as starting material. ¹H-NMR (DMSO-d₆): δ 8.76 (s, 1H), 8.15 (d, 1H), 7.48 (d, 1H), 7.37 (s, 1H), 7.19 (d, 1H), 7.05 (d, 1H), 6.14 (s, 2H), 4.42 (s, 2H), 2.36 (s, 3H).

### Example 7

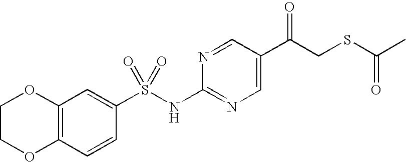

### Thioacetic acid S-{2-[2-(2,3-dihydro-benzo[1,4]dioxine-6-sulfonylamino)-pyrimidin-5-yl]-2-oxo-ethyl}ester

Step 1 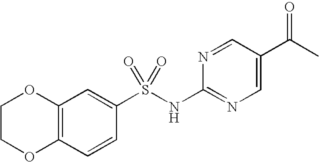
1-(2-Amino-pyrimidin-5-yl)-ethanone (0.468 g, 3.4 mmol) was dissolved in THF (18 mL) and stirred under N₂. Sodium hydride (60% in oil, 0.545 g, 13.6 mmol) was added. The mixture was heated to 60° C. for 1 hour and then cooled to room temperature. 2,3-Dihydro-benzo[1,4]dioxine-6-sulfonyl chloride (0.96 g, 4.1 mmol) was added as a solution in THF (4 mL). The reaction was stirred at room temperature overnight and then poured into water (200 mL). The aqueous solution was washed with CH₂Cl₂ (2×100 mL), acidified to pH<2 with 37% HCl, then extracted with CH₂Cl₂ (6×100 mL). The organic extracts were combined, washed with brine (200 ml), dried over MgSO₄, and concentrated in vacuo to give a yellow solid. The crude solid was purified by silica gel chromatography (40-65% EtOAc/Hexanes) to give the desired sulfonamide as a white solid (0.090 g, 8%). ¹H-NMR (DMSO-d₆): δ 12.25 (s, 1H), 8.97 (s, 2H), 7.44 (m, 2H), 7.02 (d, 1H), 4.29 (m, 4H), 2.48 (s, 3H).
Steps 2-3 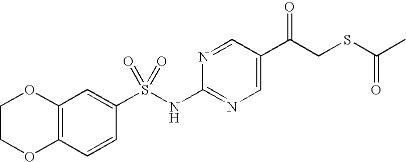
The compound was prepared according to the procedure described in Example 1 using the product of Step 1 as the starting material. ¹H-NMR (DMSO-d₆): δ 12.34 (s, 1H), 9.06 (s, 2H), 7.47 (m, 2H), 7.04 (d, 1H), 4.45 (s, 2H), 4.30 (m, 4H), 2.37 (s, 3H). LC-MS (ES+): 410 [MH]⁺ m/e.

### Example 8

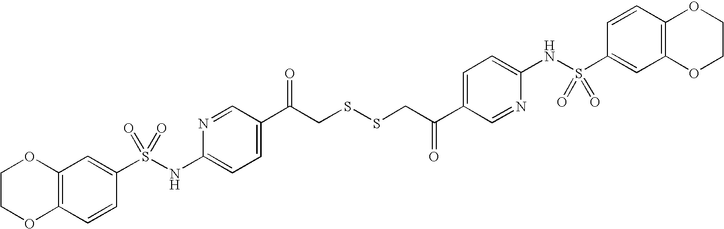 2,3-Dihydro-benzo[1,4]dioxine-6-sulfonic acid [5-(2-mercapto-acetyl)-pyridin-2-yl]-amide disulfide::Thioacetic acid S-{2-[6-(2,3-dihydro-benzo[1,4]dioxine-6-sulfonylamino)-pyridin-3-yl]-2-oxo-ethyl}ester was prepared as in Example 1 (100 mg, 0.24 mmol) was dissolved in 1N NaOH (2 mL) and stirred for 5 minutes leaving a yellow solution which was neutralized with aq. HCl. The resultant white mixture was concentrated to a white solid and suspended in methanol (2.5 mL). Methanolic I₂ was then added dropwise until no further discoloration was noted. Volatiles were removed in vacuo and the resultant residue was purified by HPLC to leave the desired compound as a white solid (40 mg, 23%). ¹H-NMR (400 MHz, DMSO-d₆): δ 11.50 (bs, 2H), 8.72 (bs, 2H), 8.14 (dd, 2H), 7.39 (m, 4H), 7.19 (bd, 2H), 7.01 (d, 2H), 4.29 (m, 12H). LC-MS (ES+): 731 [MH]⁺ m/e.

### Example 9

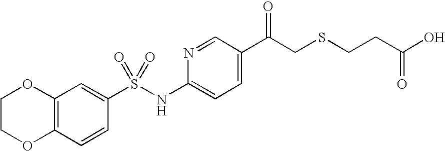 3-{2-[6-(2,3-Dihydro-benzo[1,4]dioxine-6-sulfonylamino)-pyridin-3-yl]-2-oxo-ethylsulfanyl}propionic acid: Thioacetic acid S-{2-[6-(2,3-dihydro-benzo[1,4]dioxine-6-sulfonylamino)-pyridin-3-yl]-2-oxo-ethyl}ester was prepared as in Example 1 (61 mg, 0.15 mmol), then suspended in methanol (1 mL) before 5N NaOH (0.09 mL, 0.45 mmol) was added, leaving a yellow solution which was stirred for 5 minutes. □-propiolactone (0.012 mL, 0.19 mmol) was then added, and the yellow solution was allowed to stir for 30 minutes. Volatiles were removed in vacuo and the resulting solid was purified by HPLC to afford 3-{2-[6-(2,3-Dihydro-benzo[1,4]dioxine-6-sulfonylamino)-pyridin-3-yl]-2-oxo-ethylsulfanyl}propionic acid as a white solid (25 mg, 38%). ¹H-NMR (400 MHz, DMSO-d₆): δ 8.72 (bs, 1H), 8.17 (dd, 1H), 7.40 (m, 2H), 7.19 (d, 1H), 7.01 (d, 1H), 4.29 (m, 4H), 3.96 (s, 2H) 2.63 (t, 2H), 2.52 (t, 2H). LC-MS (ES+): 439 [MH]⁺ m/e.

### Example 10

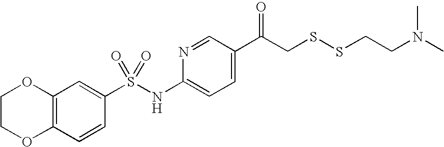 2,3-Dihydro-benzo[1,4]dioxine-6-sulfonic acid {5-[2-(2-dimethylamino-ethyldisulfanyl)-acetyl]-pyridin-2-yl}-amide: Thioacetic acid S-{2-[6-(2,3-dihydro-benzo[1,4]dioxine-6-sulfonylamino)-pyridin -3-yl]-2-oxo-ethyl}ester was prepared as in Example 1 (302 mg, 0.74 mmol) was added to dimethylaminoethane thiol hydrochloride (314 mg, 2.22 mmol) in methanol (4 mL). 5N NaOH (0.148 ml, 2.22 mmol) was added and the yellow solution was stirred for 5 minutes. The solution was neutralized with aq. HCl. Methanolic iodine was then added until discoloration was no longer apparent. Volatiles were removed in vacuo and the residue was purified by HPLC to leave the desired compound as a gum (40 mg, 12%). ¹H-NMR (400 MHz, DMSO-d₆): δ 9.61 (bs, 1H), 8.77 (bs, 1H), 8.18 (dd, 1H), 7.39 (m, 2H), 7.19 (bd, 1H), 7.02 (d, 1H), 4.38 (s, 2H) 4.29 (q, 4H), 3.43 (t, 2H), 3.03 (t, 2H) 2.79 (s, 6H). LC-MS (ES+): 470 [MH]⁺ m/e.

### Example 11

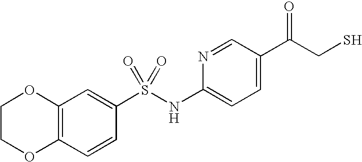 2,3-Dihydro-benzo[1,4]dioxine-6-sulfonic acid[5-(2-mercapto-acetyl)-pyridin-2-yl]-amide::Thioacetic acid S-{2-[6-(2,3-dihydro-benzo[1,4]dioxine-6-sulfonylamino)-pyridin-3-yl]-2-oxo-ethyl}ester was prepared as in Example 1 (500 mg, 1.23 mmol) was suspended in methanol (10 mL) and 5N NaOH was added (0.74 mL) affording a yellow solution. After stirring for 5 minutes, the pH was neutralized with aq. HCl, leaving an off-white mixture. Concentration in vacuo left a creme solid which was purified by HPLC to afford the desired compound (265 mg, 60%) as a white crystalline solid. ¹H-NMR (400 MHz, DMSO-d₆): δ 12.20 (bs, 1H), 8.71 (bs, 1H), 8.17 (dd, 1H), 7.39 (m, 2H), 7.20 (d, 1H), 7.01 (d, 1H), 4.29 (q, 4H), 3.98 (d, 2H), 2.91 (t, 1H). LC-MS (ES+): 367 [MH]⁺ m/e.

### Example 12

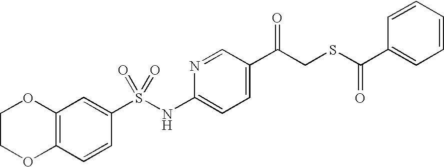 Thiobenzoic acid S-{2-[6-(2,3-dihydro-benzo[1,4]dioxine-6-sulfonylamino)-pyridin-3-yl]-2-oxo-ethyl}ester: The compound from Example 1, Step 6 (377 mg, 0.91 mmol) was suspended in methanol (2 mL) before thiobenzoic acid was added (0.107 mL, 0.91 mmol). 5N NaOH (0.183 ml, 0.91 mmol) was then added, leaving a yellow solution which was stirred for 30 minutes. Volatiles were removed in vacuo and the resulting yellow residue was purified by HPLC to afford the desired product as a white powder (255 mg, 60%). ¹H-NMR (400 MHz, DMSO-d₆): δ 8.86 (bs, 1H), 8.24 (dd, 1H), 7.94 (dd, 2H), 7.72 (m, 1H), 7.58 (m, 2H), 7.40 (bs, 2H), 7.22 (bs, 1H) 7.02 (1H), 4.67 (s, 2H), 4.30 (q, 4H). LC-MS (ES+): 471 [MH]⁺ m/e.

### Example 13

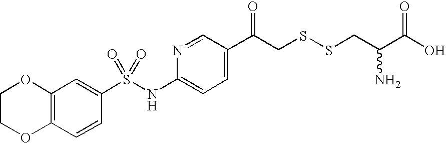 2-Amino-3-{2-[6-(2,3-dihydro-benzo[1,4]dioxine-6-sulfonylamino)-pyridin-3-yl]-2-oxo-ethyldisulfanyl}-propionic acid::Thioacetic acid S-{2-[6-(2,3-dihydro-benzo[1,4]dioxine-6-sulfonylamino)-pyridin-3-yl]-2-oxo-ethyl} ester was prepared as in Example 1 (100 mg, 0.24 mmol) was suspended in methanol (2 mL) before 5N NaOH was added (0.37 mL, 0.73 mmol) affording a yellow solution. After stirring for 5 minutes, (D,L)-cysteine (119 mg, 0.98 mmol) was added and the pH was neutralized with aq. HCl leaving an off-white mixture. Methanolic iodine was then added until there was no further discoloration. Volatiles were removed in vacuo and the residue was purified by HPLC to give the desired compound as a white powder (20 mg, 17%). ¹H-NMR (400 MHz, DMSO-d₆): δ 8.77 (bs, 1H), 8.35 (bs, 3H), 8.17 (dd, 1H), 7.39 (m, 2H), 7.21 (bs, 1H), 7.02 (d, 1H), 6.54 (bs, 1H), 4.39 (bs, 3H), 4.29 (m, 6H), 3.26 (m, 2H), 3.12 (m, 2H). LC-MS (ES+): 486 [MH]⁺ m/e.

### Example 14

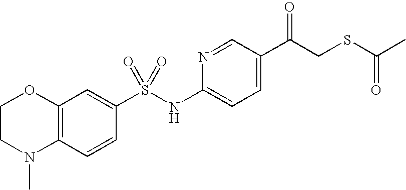 Thioacetic acid S-{2-[6-(4-methyl-3,4-dihydro-2H-benzo[1,4]oxazine-7-sulfonylamino)-pyridin-3-yl]-2-oxo-ethyl}ester: The compound was prepared according to the procedure described in Example 1 using 4-methyl-3,4-dihydro-2H-benzo[1,4]oxazine-7-sulfonyl chloride. ¹H-NMR (400 MHz, CDCl₃): δ 8.80 (s, 1H), 8.34 (d, 1H), 7.63(d, 1H), 7.24 (d, 1H), 7.19 (s, 1H), 6.80 (d, 1H), 4.31 (t, 2H), 4.21 (s, 2H), 3.30 (t, 2H), 2.94 (s, 3H), 2.41 (s, 3H). MS: (421.1).

### Example 15

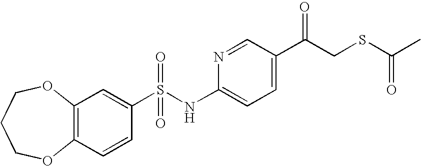

### Thioacetic acid S-{2-[6-(3,4-dihydro-2H-benzo[b][1,4]dioxepine-7-sulfonylamino)-pyridin-3-yl]-2-oxo-ethyl}ester

Step 1

3,4-Dihydro-2H-benzo[b][1,4]dioxepine: To a solution of procatechol (15 g, 136.2 mmol) in DMF (150 mL) was added K₂CO₃ (47 g, 340.6 mmol) and 1,3-dibromopropane (30.5 g, 151.1 mmol). The resulting solution was stirred for 3 h at room temperature. Water (1500 mL) was added and the mixture was extracted from EtOAc (3×300 mL). The combined organic solution was washed with NaOH/H₂O (3×300 mL), dried and concentrated under reduced pressure to give the desired compound, 19 g (91%), as a brown liquid.

Step 2

3,4-Dihydro-2H-benzo[b][1,4]dioxepine-7-sulfonyl chloride: A solution of chlorosulfonic acid (24 g, 206.0 mmol) and 3,4-dihydro-2H-benzo[b][1,4]dioxepine (10 g, 66.58 mmol) was kept at 0° C. for 30 min. The reaction mixture was poured into 1000 mL of ice/H₂O and extracted from EtOAc (3×100 mL). The combined organic layers were concentrated under reduced pressure. The residue was purified by silica gel chromatography (1:20 EtOAc/hexanes) to give the desired compound, 3.0 g (18%), as a white solid

Step 3

The compound was prepared according to the procedure described in Example 1 using 3,4-dihydro-2H-benzo[b][1,4]dioxepine-7-sulfonyl chloride. ¹H NMR (400 MHz, DMSO-d₆) δ 8.76 (s, 1H), 8.18 (d, 1H), 7.49-7.45 (m, 2H), 7.23 (d, 1H), 7.09 (d, 1H), 4.42 (s, 2H), 4.22 (m, 4H), 2.36 (s, 3H), 2.13 (m, 2H). LCMS: 423 (M+1)⁺.

### Example 16

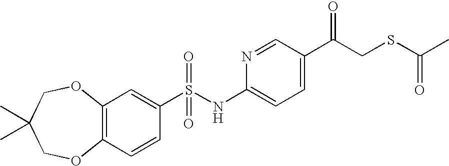 Thioacetic acid S-{2-[6-(3,3-dimethyl-3,4-dihydro-2H-benzo[b][1,4]dioxepine-7-sulfonylamino)-pyridin -3-yl]-2-oxo-ethyl}ester: The compound was prepared according to the procedure described in Example 15 using 3,3-dimethyl-3,4-dihydro-2H-benzo[b][1,4]dioxepine-7-sulfonyl chloride. ¹H NMR (400 MHz, CDCl₃) δ 9.01 (s, 1H), 8.22 (d, 1H), 7.47-7.43 (m, 3H), 6.97 (d, 1H), 4.22 (s, 2H), 3.92 (s, 2H), 3.90 (s, 2H), 2.38 (s, 3H), 1.04 (s, 6H). LCMS: 451 (M+1)⁺.

### Example 17

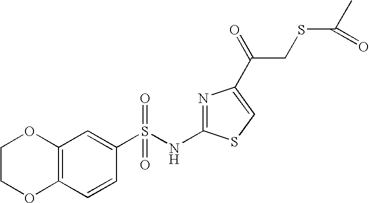 Step 1
1-Bromobutane-2,3-dione: To a solution of butane-2,3-dione (30 g, 348.84 mmol) in CCl₄ (50 mL) was added a solution of bromine (10 g, 62.9 mmol) in CCl₄ (50 mL) dropwise over 2.5 h. The resulting solution was kept at room temperature for 1 h. The reaction mixture was concentrated under reduced pressure to give the desired product.
Step 2
1-(2-Aminothiazol-4-yl)ethanone: A mixture of 1-bromobutane-2,3-dione (10.4 g, 63.41 mmol) and thiourea (1.6 g, 21.05 mmol) in EtOH (100 mL) was stirred at room temperature overnight. A solid was collected by filtration. The filter cake was washed with CH₂Cl₂ (2×100 mL) and dried to yield the desired compound, 2.0 g (67%), as a light yellow solid.
Step 3
Thioacetic acid S-{2-[2-(2,3-dihydro-benzo[1,4]dioxine-6-sulfonylamino)-thiazol-4-yl]-2-oxo-ethyl}ester: The compound was prepared according to the procedure described in Example 1 using 1-(2-aminothiazol-4-yl)ethanone. ¹H NMR (400 MHz, CDCl₃) δ 7.59 (s, 1H), 7.42-7.37 (m, 2H), 6.89 (d, 1H), 4.27 (m, 4H), 4.09 (s, 2H), 2.39 (s, 3H). LCMS: 415 (M+1)⁺.

### Example 18

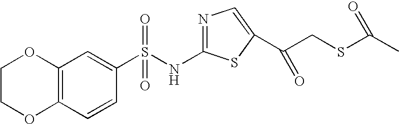 Step 1
Sodium 3-oxobut-1-en-1-olate: To a solution of sodium methoxide (27.0 g, 499.9 mmol) in ether (275 mL) was added a solution of ethyl formate (37.0 g, 499.3 mmol) in acetone (29.0 g, 483.3 mmol) dropwise over 25 min. The reaction mixture was stirred at room temperature for 15 min and a solid was collected by filtration. The filter cake was washed with ether (3×100 mL) and dried to give the desired compound, 35 g (65%), as a white solid.
Step 2
(Z)-3-Bromo-4-hydroxybut-3-en-2-one: To a solution of sodium 3-oxobut-1-en-1-olate (10 g, 92.5 mmol) in CH₂Cl₂ (120 mL) at −70° C. was added a solution of bromine (9 g, 56.3 mmol) in CH₂Cl₂ (20 mL) dropwise over 25 min. The reaction solution was stirred for 4.5 h at −70° C. Solids were removed by filtration and the filtrate was concentrated under reduced pressure to give the desired compound, 1.2 g (8%), as a yellow solid.
Step 3
1-(2-Aminothiazol-5-yl)ethanone hydrobromide: To a solution of (Z)-3-bromo-4-hydroxybut-3-en-2-one (7.54 g, 45.7 mmol) in acetone (320 mL) at 0° C. was added thiourea (3.48 g, 45.72 mmol). The reaction mixture was stirred for 20 h at room temperature and then heated to 70° C. for 1 h. The reaction mixture was cooled and a solid was collected by filtration to give the desired product, 5 g (42%) as a pale yellow solid.
Step 4

Thioacetic acid S-{2-[2-(2,3-dihydro-benzo[1,4]dioxine-6-sulfonylamino)-thiazol-5-yl]-2-oxo-ethyl}ester: The compound was prepared according to the procedure described in Example 1 using 1-(2-aminothiazol-5-yl)ethanone. ¹H NMR (400 MHz, DMSO-d₆) δ 8.54 (s, 1H), 7.30 (d, 1H), 7.23 (s, 1H), 7.01 (d, 1H), 4.29 (m, 4H), 4.28 (s, 2H), 2.37 (s, 3H). LCMS: 415 (M+1)⁺.

### Example 19

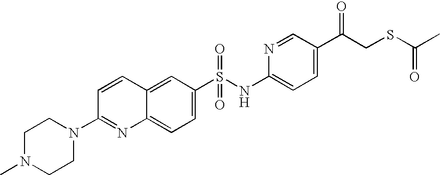

### Thioacetic acid S-(2-{6-[2-(4-methyl-piperazin-1-yl)-quinoline-6-sulfonylamino]-pyridin-3-yl}-2-oxo-ethyl)ester

Step 1

3-Chloro-N-phenylpropanamide: To a solution of aniline (9.3 g, 100.0 mmol) in acetone (100 mL) was added potassium carbonate (20.8 g, 150.7 mmol) and water (200 mL). To the mixture was added 3-chloropropanoyl chloride (15.9 g, 125.2 mmol) dropwise with stirring, while cooling to 0° C. The resulting solution was stirred for 1 h while the temperature was maintained at 0° C. The reaction mixture was then quenched by adding 500 mL of H₂O/ice. The solid precipitate was collected by filtration and dried under reduced pressure. To afford the desired compound, 18.3 g (98%), as a white solid.

Step 2

3,4-Dihydroquinolin-2(1H)-one: To a solution of 3-chloro-N-phenylpropanamide (18.3 g, 98.0 mmol) in chlorobenzene (1000 mL) was added AlCl₃ (80 g, 601.5 mmol) in small portions while cooling to 0° C. The resulting solution was heated at 120° C. for 6 h. The reaction solution was cooled, diluted with 2000 mL of H₂O/ice and extracted from CH₂Cl₂ (3×1.2 L). The combined organic solution was concentrated under reduced pressure. The residue was purified by silica gel chromatography (1:1 EtOAc/hexane) to give the desired compound, 7.2 g (47%), as a white solid.

Step 3

6-Nitro-3,4-dihydroquinolin-2(1H)-one: To a solution of 3,4-dihydroquinolin-2(1H)-one (7.2 g, 46.5 mmol) in H₂SO₄ (150 mL) at 0° C. was added water (35 ml) dropwise with stirring. To the reaction solution was added HNO₃ (3.5 mL) dropwise with stirring, while cooling to a temperature of 0° C. The resulting solution was stirred for 15 min at 0° C. The reaction mixture was then quenched by adding 350 mL of H₂O/ice. The resulting solution was extracted from EtOAc (5×250 mL). The combined organic layers were concentrated under reduced pressure to afford the desired product, 8.2 g (90%), as a yellow solid.

Step 4

2-Chloro-6-nitroquinoline: To a solution of 6-nitro-3,4-dihydroquinolin-2(1H)-one (8.2 g, 41.9 mmol) in benzene (150 mL) was added DDQ (9.6 g, 42.5 mmol) followed by dropwise addition of POCl₃ (20.5 mL). The resulting solution was heated at 90° C. for 3 h. The reaction mixture was cooled to room temperature then quenched by adding 500 mL of H₂O/ice. The pH was adjusted to 7 by the addition of 4N NaOH. The resulting solution was extracted from EtOAc (3×1 L). The combined organic layers were concentrated under reduced pressure to yield the desired product, 8.4 g (95%), as a yellow solid.

Step 5

2-Chloroquinolin-6-amine: To 2-chloro-6-nitroquinoline (8.4 g, 39.6 mmol) and NH₄Cl (6.5 g, 121.50 mmol) was added EtOH (100 mL) and water (20 mL). The reaction mixture was heated to 60° C. and Fe (10 g, 178.6 mmol) was added in several portions. The reaction mixture was stirred for 2 h maintaining the temperature at 60° C. The mixture was cooled to room temperature and the ethanol was removed under reduced pressure. The aqueous mixture was diluted with 100 mL of EtOAc and solids were removed by filtration. The filtrate was concentrated under reduced pressure to yield the desired product, 6.8 g (95%), as a yellow solid.

Step 6

2-Chloroquinoline-6-sulfonyl chloride: To a solution of 2-chloroquinolin-6-amine (1.0 g, 5.1 mmol) in acetonitrile (50 mL) at 0° C. was added acetic acid (3.3 g, 54.9 mmol) dropwise with stirring over 5 min. To the 0° C. solution was added conc HCl (2 g, 20.3 mmol) dropwise with stirring over 5 min followed by a solution of sodium nitrite (400 mg, 5.7 mmol) in water (1 mL), dropwise with stirring over 10 min. To the cold mixture was introduced sulfur dioxide (0.5 kg, 7.8 mol), while maintaining a temperature of 0° C. over 2 h. To the reaction mixture was added copper(II) chloride dihydrate (900 mg, 5.2 mmol) over 15 min while maintaining a temperature of 0° C. The reaction solution was kept at 0° C. for 50 min and warmed to room temperature overnight. The reaction mixture was then quenched with the addition of 200 mL of H₂O/ice. The resulting solution was extracted from CH₂Cl₂ (3×200 mL). The combined organic layers were washed with water water (3×200 mL), dried and concentrated under reduced pressure. The residue was purified by silica gel chromatography (1:5 EtOAc/hexanes) to afford the desired compound, 0.6 g (43%), as a yellow solid.

Step 7

N-(5-Acetylpyridin-2-yl)-2-chloroquinoline-6-sulfonamide: The compound was prepared according to the procedure described in Example 1, Step 5 using 2-chloroquinoline-6-sulfonyl chloride. ¹H NMR (400 MHz, DMSO-d₆) δ 10.93 (s, 1H), 7.96 (d, 2H), 7.84 (d, 2H), 7.57 (d, 2H), 7.20 (d, 2H), 2.46 (s, 3H). LCMS: 389 (M+1)⁺.

Step 8

2-(4-Methyl-piperazin-1-yl)-quinoline-6-sulfonic acid (5-acetyl-pyridin-2-yl)-amide: A solution of N-(5-acetylpyridin-2-yl)-2-chloroquinoline-6-sulfonamide (400 mg, 1.1 mmol) and 1-methylpiperazine (0.250 mL, 2.2 mmol) in dimethylacetamide (1.2 mL) was heated to 110° C. for 2 h. The mixture was cooled and partitioned between CH₂Cl₂ (40 mL) and a pH 8.2 aqueous phosphate buffer (2.8M, 10 mL). The organic layer was concentrated onto silica gel (2 g) and purified by flash chromatography (90 g silica gel, CH₂Cl₂ to 20% MeOH:CH₂Cl₂) to afford the desired compound, 335 mg (72%), as an off-white solid. LCMS: 426 (M+1)⁺.
Step 9 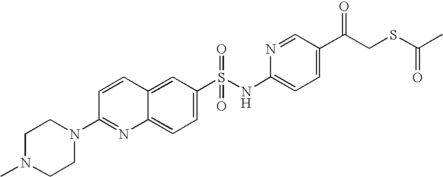
Thioacetic acid S-(2-{4-[4-(1-methyl-piperidin-4-yloxy)-benzenesulfonylamino]-phenyl}-2-oxo-ethyl) ester: The compound was prepared according to the procedure described in Example 1, Steps 6 and 7 using 2-(4-methyl-piperazin-1-yl)-quinoline-6-sulfonic acid (5-acetyl-pyridin-2-yl)-amide as starting material. ¹H NMR (400 MHz, CD₃OD, 1 eq of TFA added) δ 8.63 (s, 1H), 8.26 (s, 1H), 8.04 (t, 2H), 7.95 (d, 1H), 7.71 (d, 1H), 7.22 (d, 1H), 7.09 (d, 1H), 4.13 (s, 2H), 3.9 (m, 4H), 3.3 (m, 4H), 2.84 (s, 3H), 2.32 (s, 3H). LCMS: 500 (M+1)⁺.

### Example 20

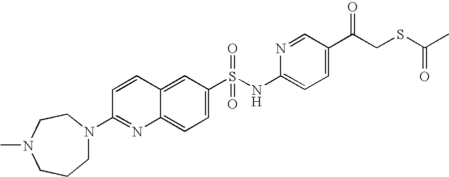 Thioacetic acid S-(2-{6-[2-(4-methyl-[1,4]diazepan-1-yl)-quinoline-6-sulfonylamino]-pyridin-3-yl}-2-oxo-ethyl)ester: The compound was prepared according to the procedure described in Example 19 using 2-chloro-quinoline-6-sulfonic acid (5-acetyl-pyridin-2-yl)-amide and 1-methylhomopiperazine as starting materials. LCMS: 514 (M+1)⁺.

### Example 21

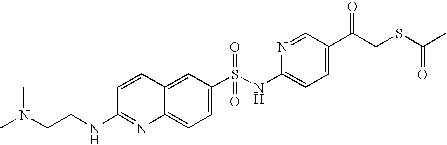 Thioacetic acid S-(2-{6-[2-(2-dimethylamino-ethylamino)-quinoline-6-sulfonylamino]-pyridin-3-yl}-2-oxo-ethyl)ester: The compound was synthesized as described in Example 19 using 2-chloro-quinoline -6-sulfonic acid (5-acetyl-pyridin-2-yl)-amide and N¹,N¹-dimethyl-ethane-1,2-diamine as starting materials. LCMS: 488 (M+1)⁺.

### Example 22

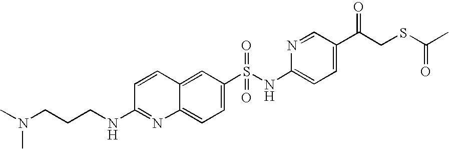 Thioacetic acid S-(2-{6-[2-(3-dimethylamino-propylamino)-quinoline-6-sulfonylamino]-pyridin-3-yl}-2-oxo-ethyl)ester: The compound was synthesized as described in Example 19 using 2-chloro-quinoline -6-sulfonic acid (5-acetyl-pyridin-2-yl)-amide and N¹,N¹-dimethyl-propane-1,3-diamine as starting materials. LCMS: 502 (M+1)⁺.

### Example 23

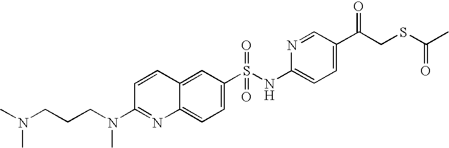 Thioacetic acid S-[2-(6-{2-[(3-dimethylamino-propyl)-methyl-amino]-quinoline-6-sulfonylamino}-pyridin-3-yl)-2-oxo-ethyl]ester: The compound was synthesized as described in Example 19 using 2-chloro-quinoline-6-sulfonic acid (5-acetyl-pyridin-2-yl)-amide and N,N,N¹-trimethyl-propane-1,3-diamine as starting materials. LCMS: 516 (M+1)⁺.

### Example 24

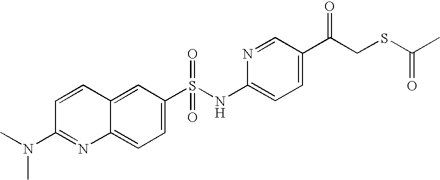 Thioacetic acid S-{2-[6-(2-dimethylamino-quinoline-6-sulfonylamino)-pyridin-3-yl]-2-oxo-ethyl}ester: The compound was synthesized as described in Example 19 using 2-chloro-quinoline-6-sulfonic acid (5-acetyl-pyridin-2-yl)-amide and dimethylamine as starting materials. LCMS: 445 (M+1)⁺.

The following preferred compounds can be made using the methods as described above and when made should have similar activity as those made above. Substitutents are defined as in Formula VI if they are not listed below.

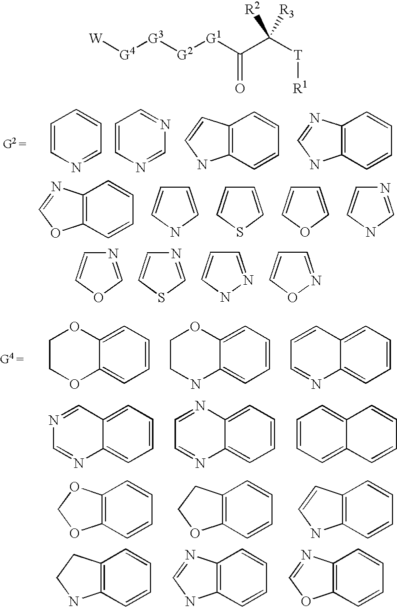

The following prophetic structures are illustrative of some of other possible combinations, including optional substitutions:

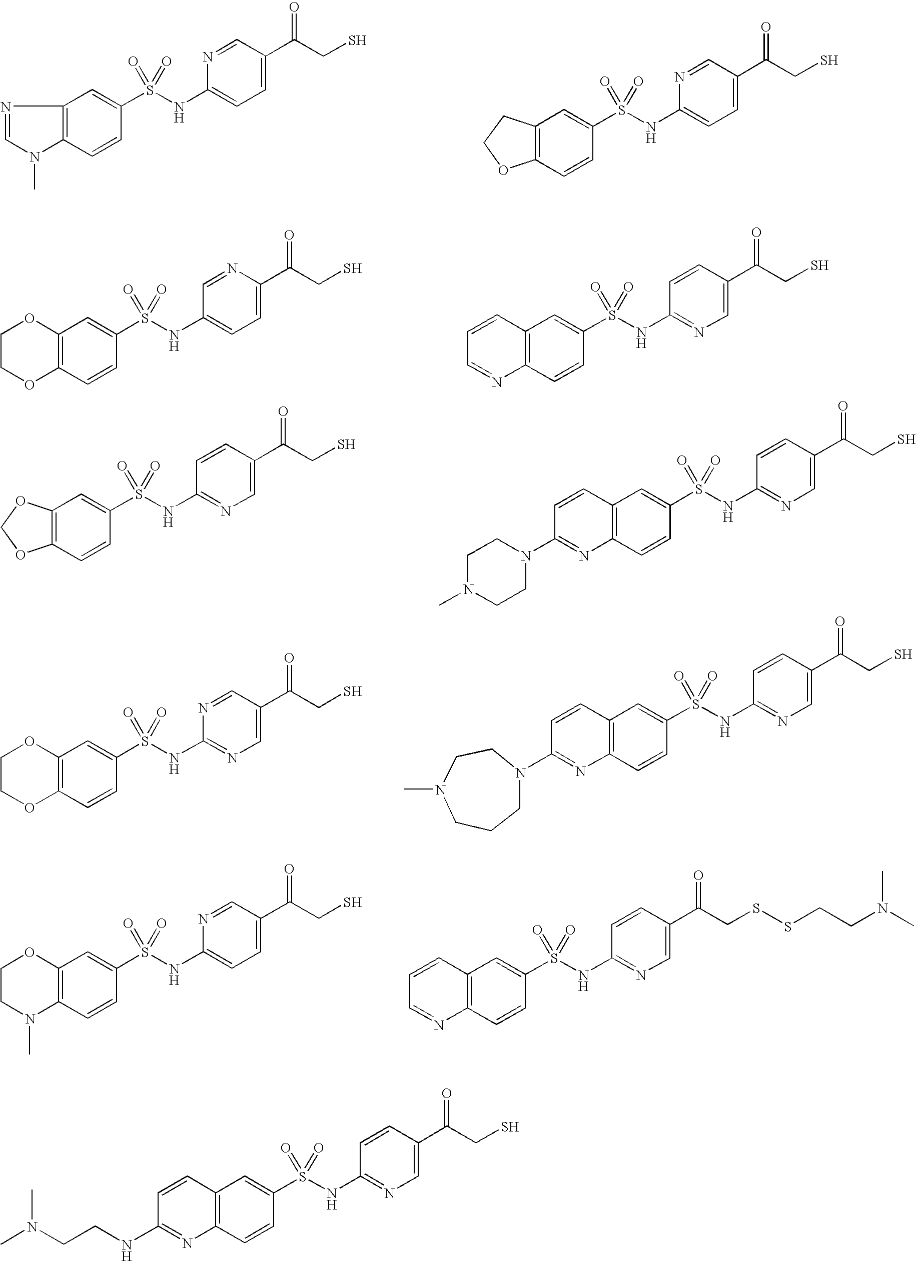

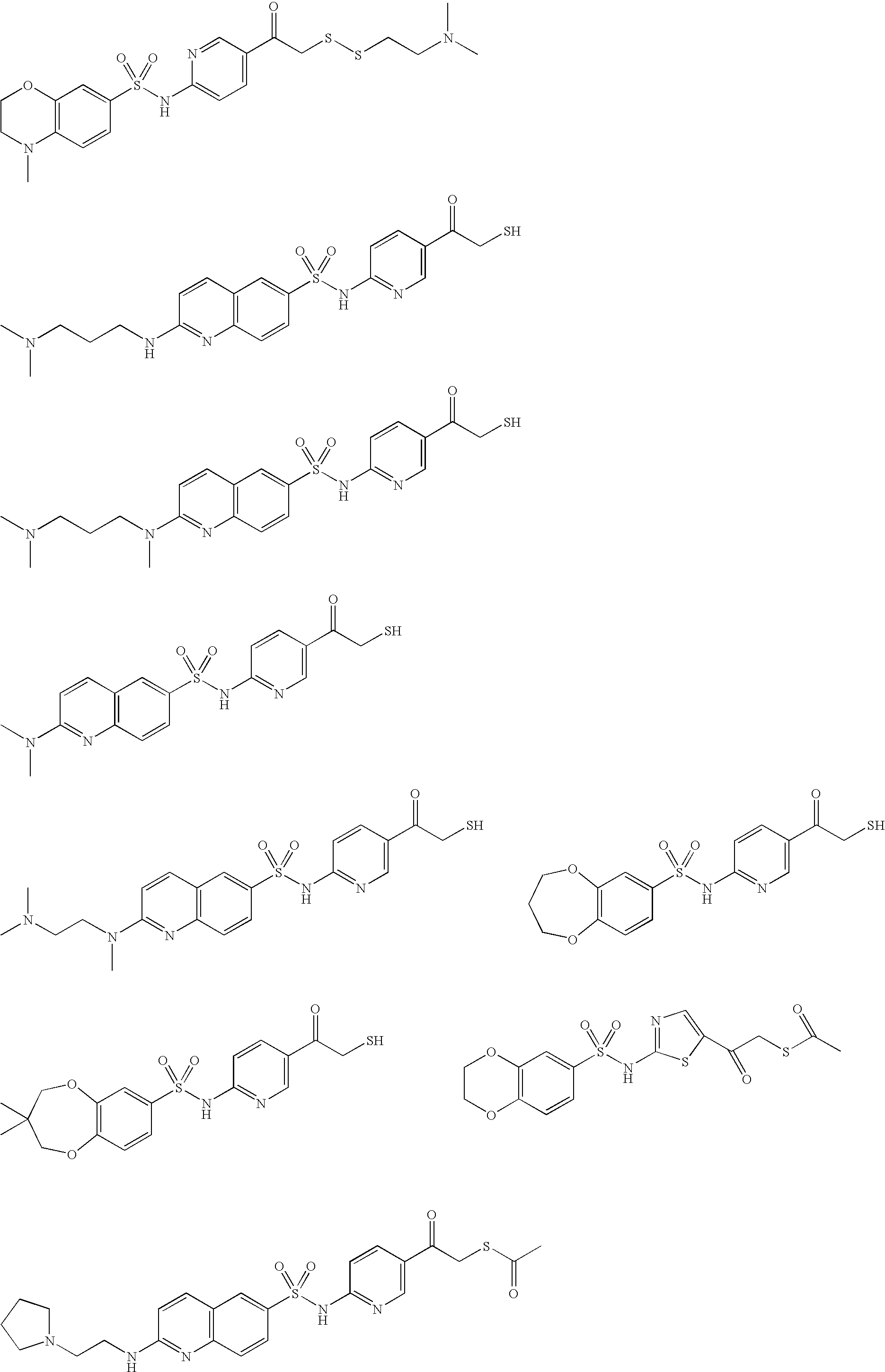

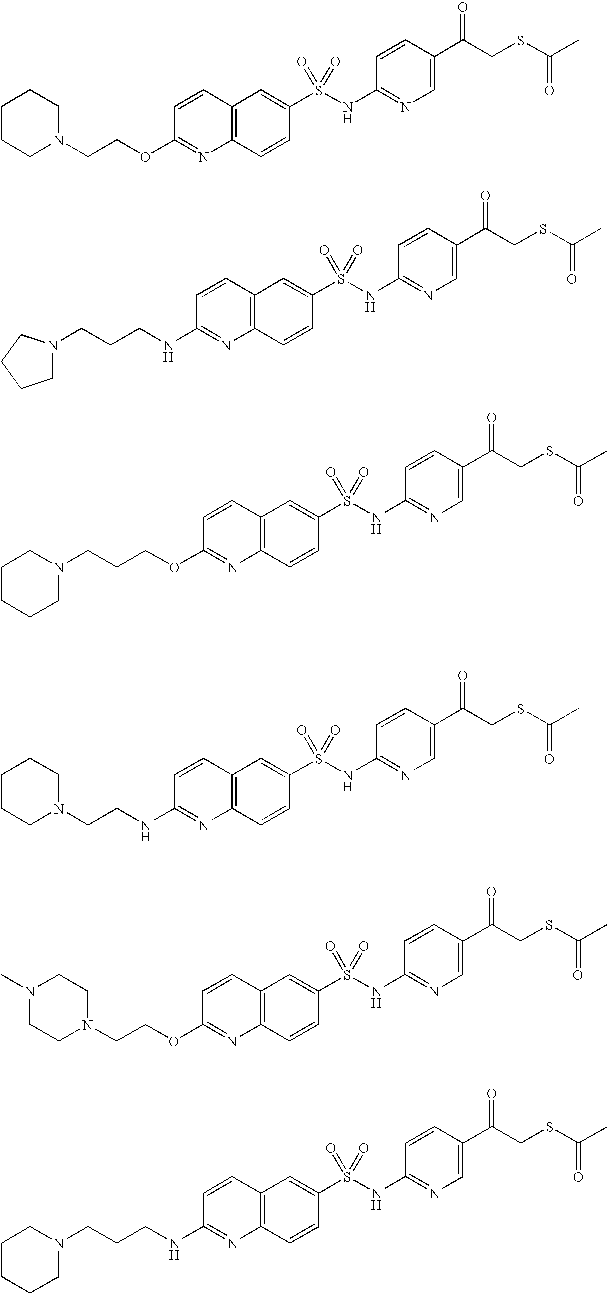

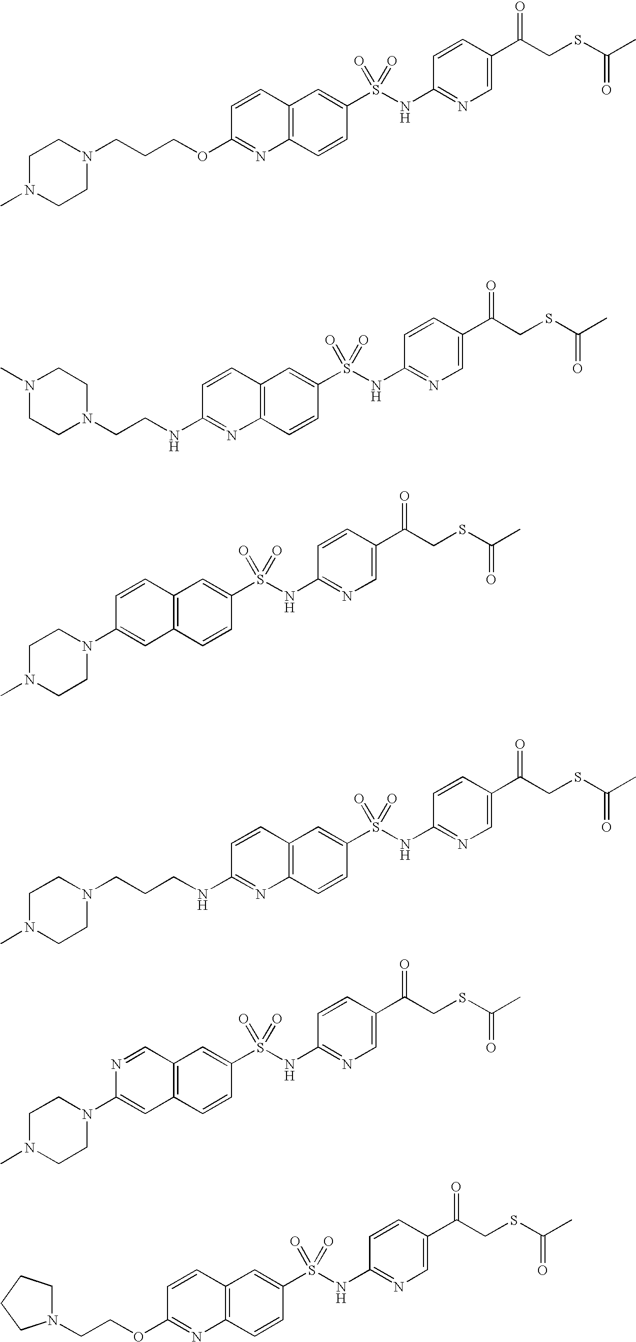

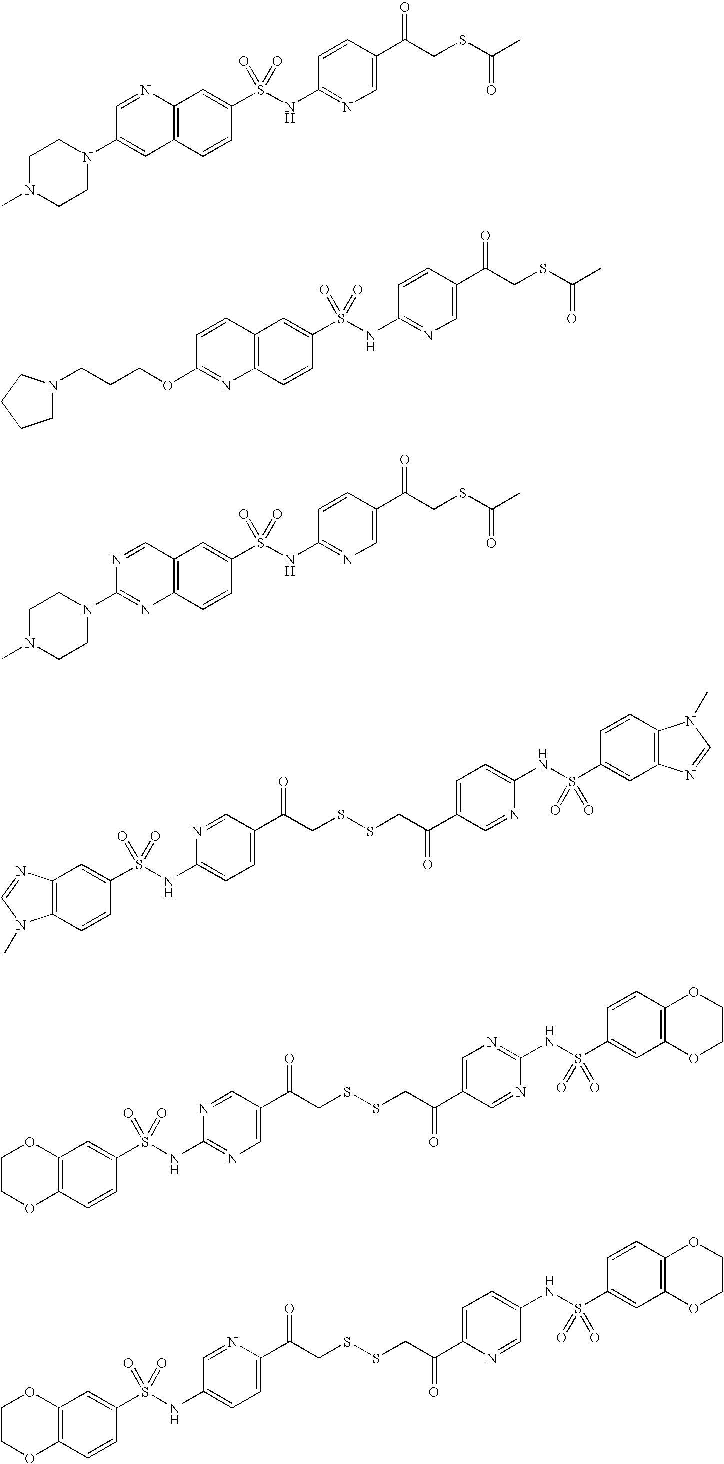

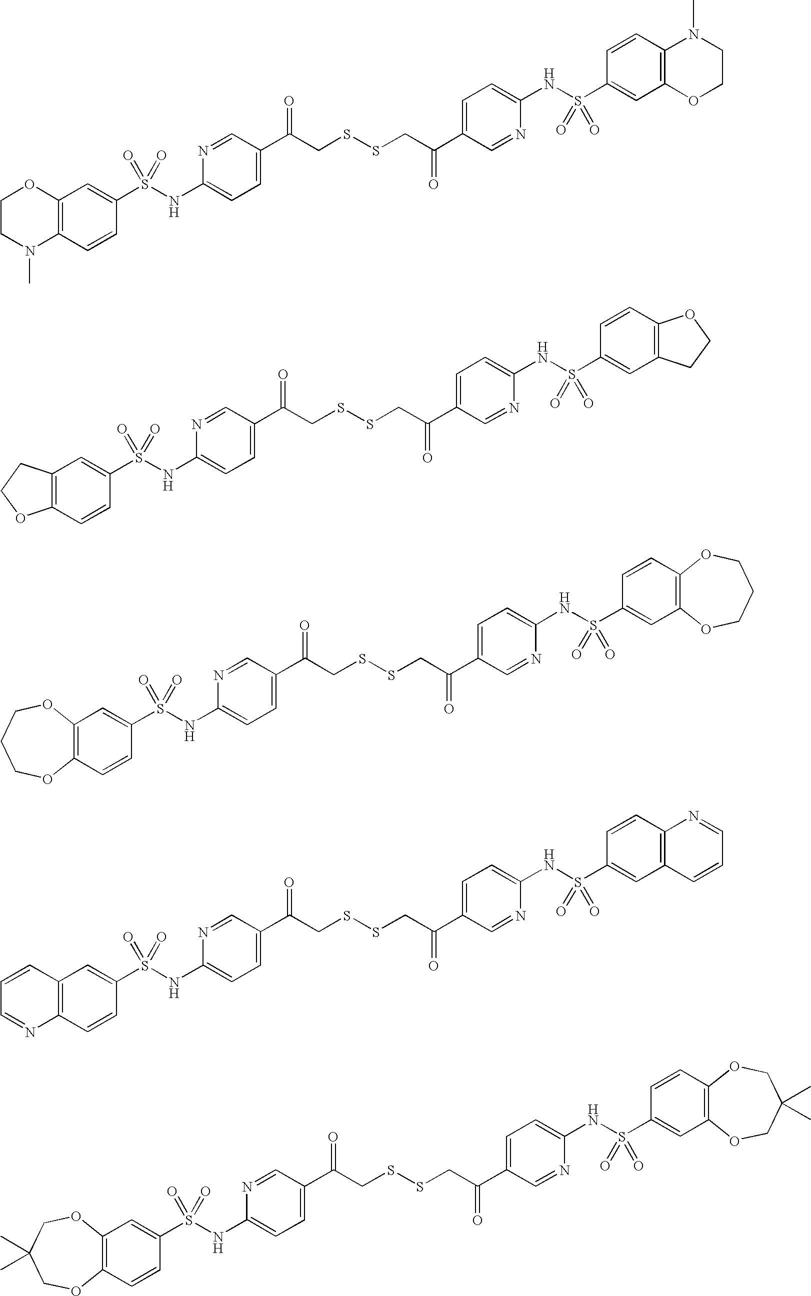

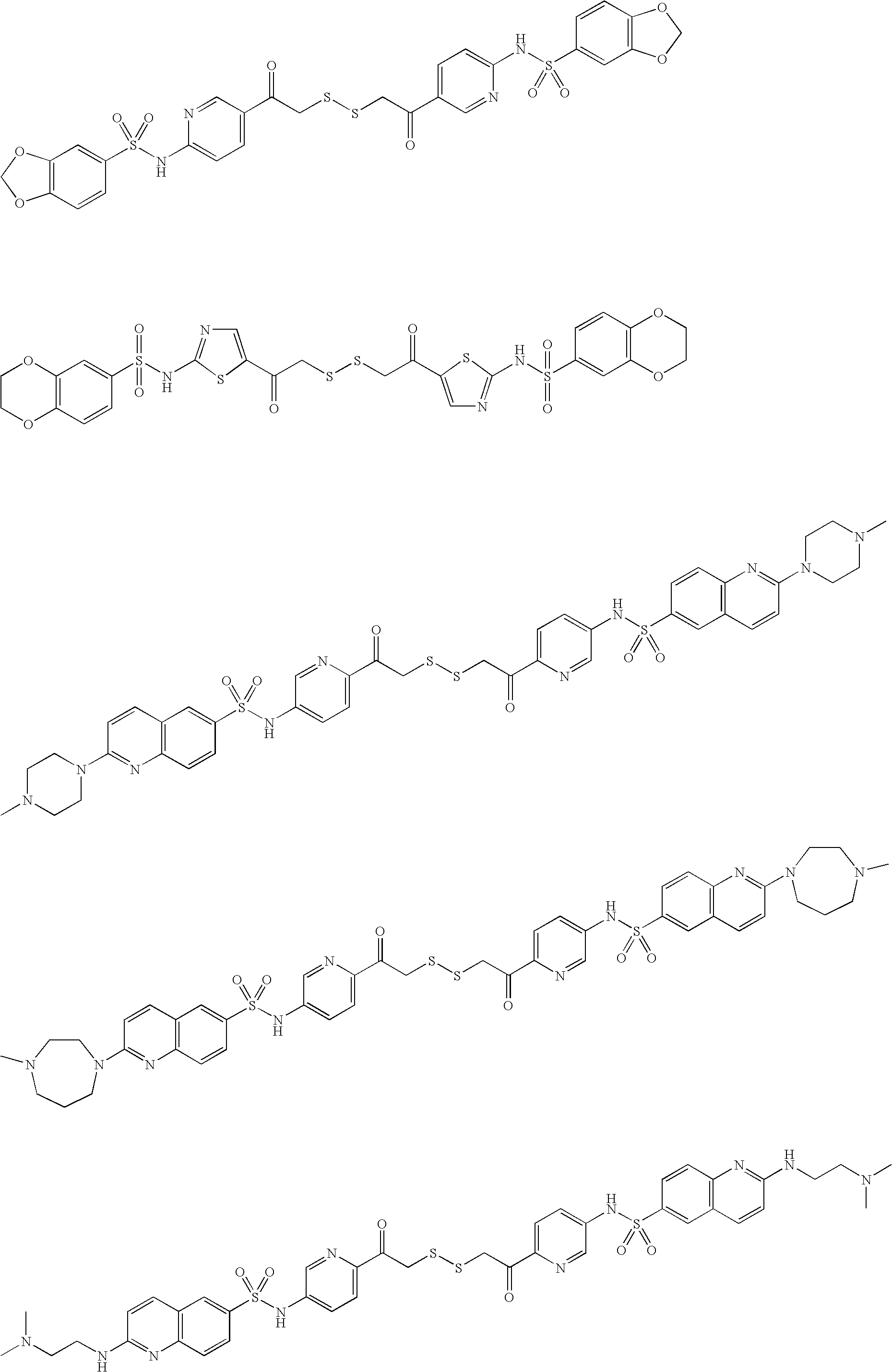

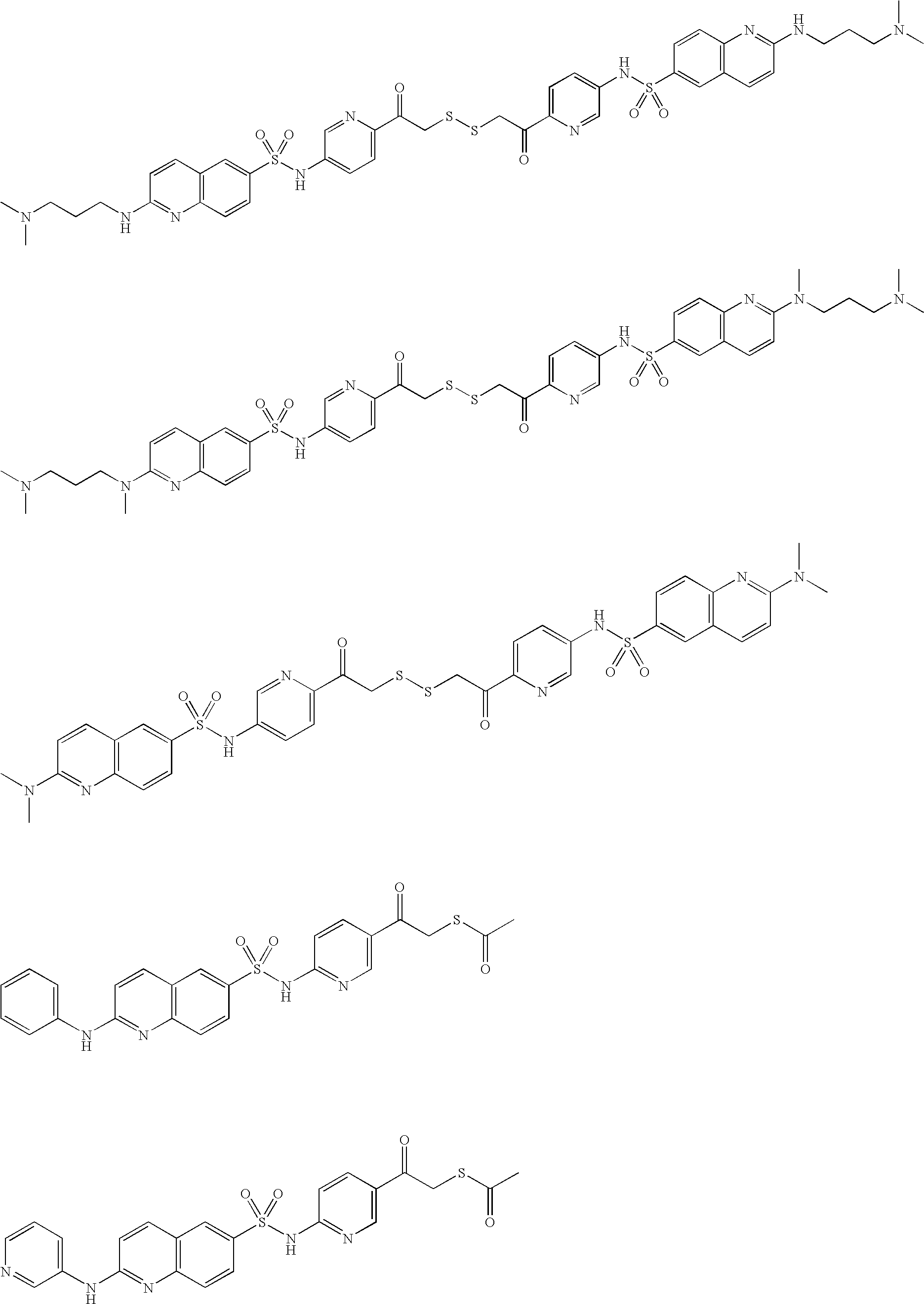

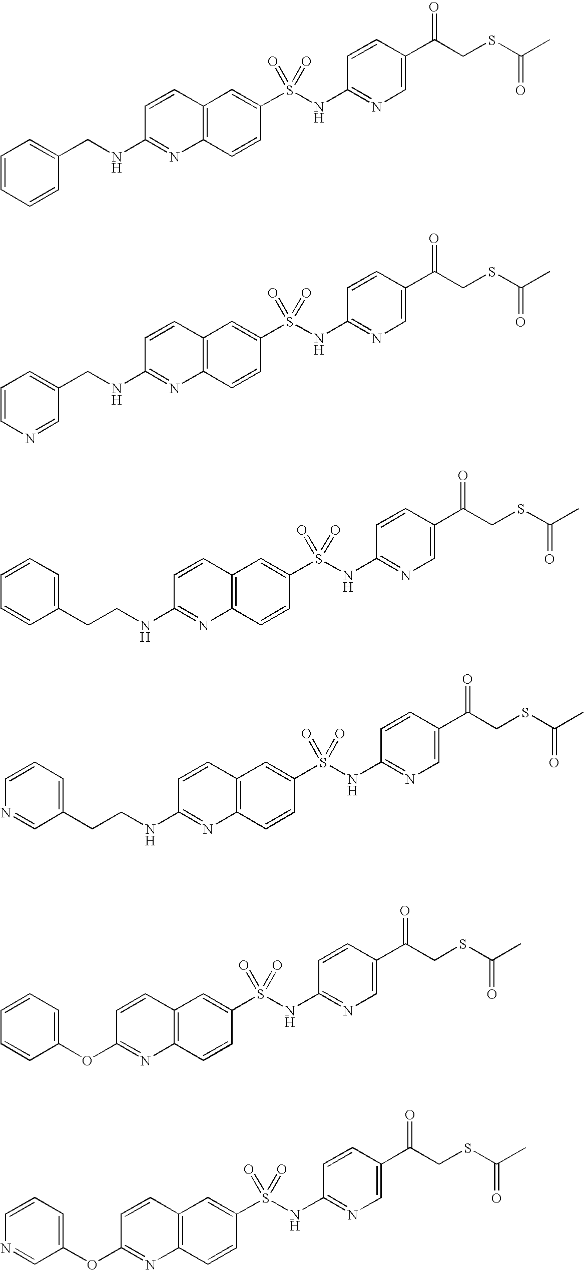

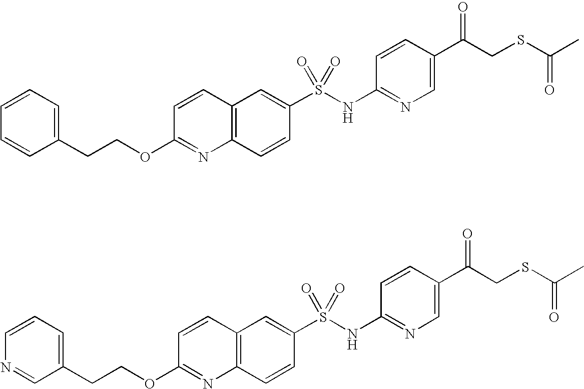

The activity of the above mentioned compounds as HDAC inhibitors has generally been shown by the following assays. The other compounds listed above, which may not yet been made or tested, are predicted to generally have activity in these assays as well.

### Inhibition Assays

### In Vitro HDAC-Inhibition Assay

This assay measures a compound's ability to inhibit acetyl-lysine deacetylation in vitro and was used as both a primary screening method as well as for IC50 determinations of confirmed inhibitors. The assay is performed in vitro using an HDAC enzyme source (e.g. partially purified nuclear extract or immunopurified HDAC complexes) and a proprietary fluorescent substrate/developer system (HDAC Quantizyme Fluor de Lys Fluorescent Activity Assay, BIOMOL). The assay is run in 1,536-well Greiner white-bottom plates using the following volumes and order of addition:

Step 1: Enzyme (2.5 ul) source added to plate (from refrigerated container)

Step 2: Compounds (50 nl) added with pin transfer device

Step 3: Fluor de Lys (2.5 ul) substrate added, incubate at RT, 30 minutes

Step 4: Developer (5 ul) solution is added (containing TSA), to stop reaction

Step 5: Plate Reader—data collection

The deacetylated fluorophore is excited with 360 nm light and the emitted light (460 nm) is detected on an automated fluorometric plate reader (Aquest, Molecular Devices).

### Cellular Histone Hyperacetylation Assays

These two secondary assays evaluates a compound's ability to inhibit HDAC in cells by measuring cellular histone acetylation levels. The cytoblot facilitates quantitative EC50 information for cellular HDAC inhibition. Transformed cell lines (e.g. HeLa, A549, MCF-7) are cultured under standard media and culture conditions prior to plating.

For Cytoblot:

Cells (approx. 2,500/well) are allowed to adhere 10-24 hours to wells of a 384-well Greiner PS assay plate in media containing 1-5% serum. Cells are treated with appropriate compound and specific concentrations for 0 to 24 hours. Cells are washed once with PBS (60 ul) and then fixed (95% ethanol, 5% acetic acid or 2% PFA) for 1 minute at RT (30 ul). Cells are blocked with 1% BSA for 1 hour and washed and stained with antibody (e.g. anti-Acetylated Histone H3, Upstate Biotechnology), followed by washing and incubation with an appropriate secondary antibody conjugated to HRP or fluorophore. For luminescence assays, signal is generated using Luminol substrate (Santa Cruz Biotechnology) and detected using an Aquest plate reader (Molecular Devices).

For Immunoblot:

Cells (4×10ˆ5/well) are plated into Corning 6-well dish and allowed to adhere overnight. Cells are treated with compound at appropriate concentration for 12-18 hours at 37 degrees. Cells are washed with PBS on ice. Cells are dislodged with rubber policeman and lysed in buffer containing 25 mM Tris, pH7.6; 150 mM NaCl, 25 mM MgCl2, 1% Tween-20, and nuclei collected by centriguation (7500 g). Nuclei are washed once in 25 mM Tris, pH7.6; 10 mM EDTA, collected by centrifugation (7500 g). Supernatant is removed and histones are extracted using 0.4 M HCl. Samples are centrifuged at 14000 g and supernatants are precipitated in 1 ml cold acetone. The histone pellet is dissolved in water and histones are separated and analyzed by SDS-PAGE Coomassie and immunobloting (anti-acetylated histone antibodies, Upstate Biotechnology) using standard techniques.

### Differential Cytotoxicity Assay

HDAC inhibitors display differential cytotoxicity toward certain transformed cell lines. Cells are cultured according to standard ATCC recommended conditions that are appropriate to each cell type. Compounds were tested for their ability to kill different cell types (normal and transformed) using the ATPlite luminescence ATP detection assay system (Perkin Elmer). Assays are run in either 384-well or 1536-well Greiner PS plates. Cells (30 ul or 5 ul, respectively) are dispensed using either multichannel pipette for 384-well plates, or proprietary Kalypsys bulk liquid dispenser for 1536-well plates. Compounds added using proprietary pin-transfer device (500 nL or 5 nL) and incubated 5 to 30 hours prior to analysis. Luminescence is measured using Aquest plate reader (Molecular Devices).

The activity of some of the compounds of the invention are shown in Table 1.

TABLE 1

| | In vitro IC₅₀ (μM) | Cellular IC₅₀ (μM) |
|---|---|---|
| | + indicates ≦1 | + indicates ≦1 |
| Example No. | − indicates >1 | − indicates >1 |
| 1 | + | + |
| 2 | + | + |
| 3 | + | + |
| 4 | + | + |
| 5 | + | + |
| 6 | + | + |
| 7 | + | − |
| 8 | + | + |
| 9 | − | NT |
| 10 | + | + |
| 11 | + | + |
| 12 | + | + |
| 13 | + | + |
| 14 | + | + |
| 15 | NT | + |
| 16 | NT | + |
| 17 | NT | − |
| 18 | NT | + |
| 19 | NT | + |
| 20 | NT | + |
| 21 | NT | + |
| 22 | NT | + |
| 23 | NT | + |
| 24 | NT | + |

### Dose Escalation Study

In the dose escalation study, SAHA was used as a standard. 5×10₆ HCT-116 colorectal cancer cells were injected subcutaneously into the right flank of 4-6 week old female nude (nu/nu) mice. Ten days post injection, tumors were randomized into cohorts (n=10) with a mean size of 131 mm³ (SEM: 44 mm³). Tumor bearing animals were dosed daily with Example 1 and SAHA formulated in 1.0% carboxymethylcellulose by oral gavage at 100, 150, 175, 250 mg/kg. In addition, SAHA was dosed at these 4 doses, in addition to 350 and 475 mg/kg. Tumor burden was determined twice weekly by measurement with calipers in 2-dimensions (length (l)×width (w)) and the volume of spheroid calculated using the formula (l×(w)²/2). Bodyweight was measured and recorded on same day as tumor volume measurement. Maximum tolerated dose (MTD) was the highest dose of test compound that did not result in any lethality or ≧20% bodyweight loss. MTD was exceeded for Example 1 at lowest dose tested, 100 mg/kg. However, previously, the dose of 50 mg/kg was determined to be well tolerated in that no significant weight loss or mortality was observed and hence, it follows that the T/C value for efficacy at MTD is 53%. For SAHA, lethality was observed at 350 mg/kg and therefore 250 mg/kg represents MTD in this study, with a T/C efficacy value of 52%. It follows that at the MTD, Example 1 and SAHA exhibit roughly equivalent efficacy in the HCT-116 xenograft model. The results of the dose escalation study with Example 1 and SAHA are shown in Table 2.

TABLE 2

| | Example 1 | Example 1 | SAHA | SAHA |
|---|---|---|---|---|
| Dose | (T/C) | (Survival) | (T/C) | (Survival) |
| 50 mg/kg** | 53% | 10/10 | 59% | 10/10 |
| 100 mg/kg | 42% | 8/10 | 65% | 10/10 |
| 125 mg/kg | 29% | 7/10 | 73% | 10/10 |
| 175 mg/kg | 11% | 0/10 | 41% | 10/10 |
| 250 mg/kg | 15% | 2/10 | 52% | 10/10 |
| 350 mg/kg | NA | NA | 24% | 9/10 |
| 475 mg/kg | NA | NA | 21% | 4/10 |

**Indicates data from another study. All compounds were dosed QD orally in 1.0% methylcellulose.
T/C = 100 * (final treated tumor volume − starting treated tumor volume)/(final control tumor volume − starting control tumor volume). A T/C value of 0 reflects complete tumor stasis. (n = 8-10 mice per treatment group)

All references cited above are incorporated herein by reference in their entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of Formula I 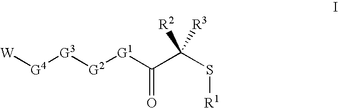
or a therapeutically acceptable salt, ester, or prodrug, thereof, wherein:
G¹ is selected from the group consisting of a bond, alkenyl, alkoxy, alkoxyalkyl, alkyl, alkylamino, alkylcarbonyl, alkylcarbonylalkyl, alkylcarbonylamino, alkylcarbonylaminoalkyl, alkynyl, amino, aminoalkyl, carbonylalkyl, and carbonylaminoalkyl;
G² is selected from the group consisting of optionally substuteted monocyclic heteroaryl, and optionally substuteted polycyclic heteroaryl;
G³ is selected from the group consisting of —X¹SO₂N(R⁷)— and —X¹N(R⁷)SO₂—;
X¹ is selected from the group consisting of a bond or an alkyl of length C₁ to C₃, any carbon atom of which may be optionally substituted;
R⁷ is selected from the group consisting of hydrogen, alkenyl, and alkyl, or alternatively, R⁷ may be joined to G² to form a heterocyclo or heteroaryl ring;
G⁴ is selected from the group consisting of bicyclic aryl, bicyclic heteroaryl, cycloalkyl-fused monocyclic aryl, cycloalkyl-fused monocyclic heteroaryl, heterocycloalkyl-fused monocyclic aryl, and heterocycloalkyl-fused monocyclic heteroaryl, wherein each may be optionally substituted;
W is selected from the group consisting of null and —U¹X²U²;
U¹ is selected from the group consisting of a bond, heterocycloalkyl, —NR¹⁰—, —O—, —S—, —C(O)N(R¹⁰), —N(R¹⁰)C(O)—, —S(O)₂N(R¹⁰), and —N(R¹⁰)S(O)—;
R¹⁰ is selected from the group consisting of hydrogen, alkenyl, and alkyl;
U² is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted;
X² is selected from the group consisting of a bond or an alkyl of length C₁ to C₇, any carbon atom of which may be optionally substituted;
R² and R³ are independently selected from the group consisting of hydrogen, methyl, and ethyl;
R¹ is selected from the group consisting of hydrogen, —P(O)(OR¹⁴)OR¹⁵, cyano, optionally substituted acyl, aroyl, aryl, alkyl, heteroaryl, heterocycloalkyl, carboxy, carboxyalkyl, optionally substituted alkylthio, optionally substituted arylthio, and a group of structural Formula II 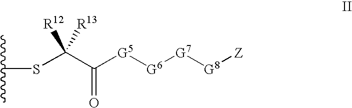
R¹⁴ and R¹⁵ are independently selected from the group consisting of hydrogen, alkyl, aryl, and heteroaryl;
R¹² and R¹³ are independently selected from the group consisting of hydrogen, methyl, and ethyl;
G⁵ are independently selected from the group consisting of a bond, alkenyl, alkoxy, alkoxyalkyl, alkyl, alkylamino, alkylcarbonyl, alkylcarbonylalkyl, alkylcarbonylamino, alkylcarbonylaminoalkyl, alkynyl, amino, aminoalkyl, carbonylalkyl, and carbonylaminoalkyl;
G⁶ are independently selected from the group consisting of optionally substuteted monocyclic heteroaryl, and optionally substuteted polycyclic heteroaryl;
G⁷ is selected from the group consisting of —X³SO₂N(R⁸)— and —X³N(R⁸)SO₂—;
X³ is selected from the group consisting of a bond or an alkyl of length C₁ to C₃, any carbon atom of which may be optionally substituted;
R⁸ is selected from the group consisting of hydrogen, alkenyl, and alkyl, or alternatively, R⁸ may be joined to G⁵ to form a heterocyclo or heteroaryl ring;
G⁸ is selected from the group consisting of bicyclic aryl, bicyclic heteroaryl, cycloalkyl-fused monocyclic aryl, cycloalkyl-fused monocyclic heteroaryl, heterocycloalkyl-fused monocyclic aryl, and heterocycloalkyl-fused monocyclic heteroaryl, wherein each may be optionally substituted;
Z is selected from the group consisting of null and —U³X⁴U⁴;
U³ is selected from the group consisting of a bond, heterocycloalkyl, —NR¹¹—, —O—, —S—, —C(O)N(R¹¹)—, —N(R¹¹)C(O)—, —S(O)₂N(R¹¹)—, and —N(R¹¹)S(O)—;
R¹¹ is selected from the group consisting of hydrogen, alkenyl, and alkyl;
U⁴ is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted; and
X⁴ is selected from the group consisting of a bond or an alkyl of length C₁ to C₇, any carbon atom of which may be optionally substituted.

2. The compound as recited in claim 1, wherein G¹ is a bond.

3. The compound as recited in claim 2, wherein R¹ is hydrogen.

4. The compound as recited in claim 2, wherein R¹ is acyl.

5. The compound as recited in claim 3 or claim 4, wherein both R² are hydrogen.

6. The compound as recited in claim 5, wherein
G³ is selected from the group consisting of —X¹SO₂N(R⁷)— and —X¹N(R⁷)SO₂—; and
X¹ is a bond.

7. The compound as recited in claim 6, wherein G² is an optionally substituted 6-membered heteroaryl.

8. The compound as recited in claim 7, wherein G⁴ is an optionally substituted napthyl.

9. The compound as recited in claim 7, wherein G⁴ is an optionally substituted bicyclic heteroaryl.

10. The compound as recited in claim 7, wherein G⁴ is an optionally substituted cycloalkyl-fused monocyclic aryl or cycloalkyl-fused monocyclic heteroaryl.

11. The compound as recited in claim 7, wherein G⁴ is a heterocycloalkyl-fused monocyclic aryl or heterocycloalkyl-fused monocyclic heteroaryl, wherein each may be optionally substituted.

12. The compound as recited in claim 6, wherein G² is an optionally substituted polycyclic heteroaryl.

13. The compound as recited in claim 12, wherein G⁴ is an optionally substituted napthyl.

14. The compound as recited in claim 12, wherein G⁴ is an optionally substituted bicyclic heteroaryl.

15. The compound as recited in claim 12, wherein G⁴ is an optionally substituted cycloalkyl-fused monocyclic aryl or cycloalkyl-fused monocyclic heteroaryl.

16. The compound as recited in claim 12, wherein G⁴ is a heterocycloalkyl-fused monocyclic aryl or heterocycloalkyl-fused monocyclic heteroaryl, wherein each may be optionally substituted.

17. A compound of structural Formula III 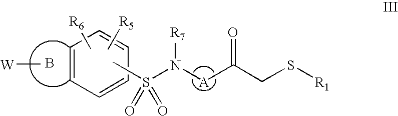
or a therapeutically acceptable salt, ester, or prodrug, thereof, wherein
A is a six-membered heteroaryl ring or polycyclic heteroaryl;
B is a saturated or unsaturated hydrocarbon chain or a saturated or unsaturated heteroatom-comprising hydrocarbon chain having from 3 to 5 atoms, forming a five- to seven-membered ring;
W is selected from the group consisting of null and —U¹X²U²;
U¹ is selected from the group consisting of a bond, heterocycloalkyl, —NR¹⁰—, —O—, —S—, —C(O)N(R¹⁰), —N(R¹⁰)C(O)—, —S(O)₂N(R¹⁰), and —N(R¹⁰)S(O)—;
U² is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted;
X² is selected from the group consisting of a bond or an alkyl of length C₁ to C₇, any carbon atom of which may be optionally substituted;
R¹ is selected from the group consisting of hydrogen, —P(O)(OR¹⁴)OR¹⁵, cyano, acyl, aryl, alkyl, heteroaryl, heterocycloalkyl and Z, wherein Z has the structural Formula IV 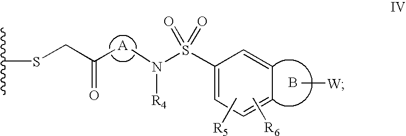
R⁴ is selected from the group consisting of hydrogen, alkenyl, and alkyl;
R¹⁴ and R¹⁵ are each independently selected from the group consisting of hydrogen, alkyl, aryl, and heteroaryl;
R⁵ and R⁶ are each independently selected from the group consisting of hydrogen, alkyl, heteroalkyl, alkenyl, alkoxy, alkoxyalkyl, cyano, halo, haloalkoxy, haloalkyl, hydroxyl, amino and nitro; and
R¹⁰ is selected from the group consisting of hydrogen, alkenyl, and alkyl.

18. The compound as recited in claim 17, wherein R¹ is hydrogen or acyl.

19. The compound as recited in claim 18, wherein A is a six-membered heteroaryl ring.

20. The compound as recited in claim 19, wherein B has the structural Formula V 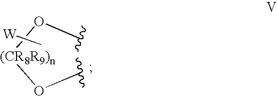
R⁸ and R⁹ are each independently selected from the group consisting of hydrogen, lower alkyl, lower alkenyl, and lower alkynyl;
n is an integer from 1 to 3; and
W is null.

21. The compound as recited in claim 20, wherein n is 2 and both R⁸ and R⁹ are hydrogen.

22. The compound as recited in claim 21, wherein R⁷ is hydrogen.

23. The compound as recited in claim 22, wherein A is a pyridyl ring.

24. The compound as recited in claim 20, wherein B has the structural Formula VI

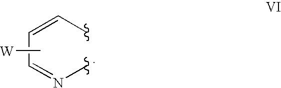

25. The compound as recited in claim 24, wherein
U¹ is selected from the group consisting of a bond, heterocycloalkyl, —NR¹⁰—, —O—, —O—; and
X² is selected from the group consisting of a bond or an alkyl of length C₁ to C₄, any carbon atom of which may be optionally substituted.

26. The compound as recited in claim 25, wherein R⁷ is hydrogen.

27. The compound as recited in claim 26, wherein A is a pyridyl ring.

28. The compound as recited in claim 1 wherein said compound is selected from the group consisting of Examples 1-24.

29. The compound as recited in claim 1 wherein said compound is Example 20.

30. The compound as recited in claim 1 wherein the compound or pharmaceutically acceptable salt, amide, ester or prodrug thereof is capable of inhibiting the catalytic activity of histone deacetylase (HDAC).

31. A pharmaceutical composition comprising a compound as recited in claim 1 together with at least one pharmaceutically acceptable carrier, diluent or excipient.

32. A method of inhibition of HDAC comprising a compound having a Formula VII: 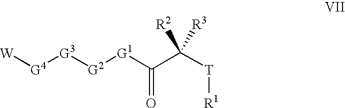
or a therapeutically acceptable salt, ester, or prodrug, thereof, wherein:
G¹ is selected from the group consisting of a bond, alkenyl, alkoxy, alkoxyalkyl, alkyl, alkylamino, alkylcarbonyl, alkylcarbonylalkyl, alkylcarbonylamino, alkylcarbonylaminoalkyl, alkynyl, amino, aminoalkyl, carbonylalkyl, and carbonylaminoalkyl;
G² is selected from the group consisting of optionally substuteted monocyclic heteroaryl, and optionally substuteted polycyclic heteroaryl;
G³ is selected from the group consisting of —X¹SO₂N(R⁷)— and —X¹N(R⁷)SO₂—;
X¹ is selected from the group consisting of a bond or an alkyl of length C₁ to C₃, any carbon atom of which may be optionally substituted;
R⁷ is selected from the group consisting of hydrogen, alkenyl, and alkyl, or alternatively, R⁷ may be joined to G² to form a heterocyclo or heteroaryl ring;
G⁴ is selected from the group consisting of bicyclic aryl, bicyclic heteroaryl, cycloalkyl-fused monocyclic aryl, cycloalkyl-fused monocyclic heteroaryl, heterocycloalkyl-fused monocyclic aryl, and heterocycloalkyl-fused monocyclic heteroaryl, wherein each may be optionally substituted;
T is selected from the group consisting of O and S;
W is selected from the group consisting of null and —U¹X²U²;
U¹ is selected from the group consisting of a bond, heterocycloalkyl, —NR¹⁰—, —O—, —S—, —C(O)N(R¹⁰)—, —N(R¹⁰)C(O)—, —S(O)₂N(R¹⁰)—, and —N(R¹⁰)S(O)—;
R¹⁰ is selected from the group consisting of hydrogen, alkenyl, and alkyl;
U² is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted;
X² is selected from the group consisting of a bond or an alkyl of length C₁ to C₇, any carbon atom of which may be optionally substituted;
R² and R³ are independently selected from the group consisting of hydrogen, methyl, and ethyl;
R¹ is selected from the group consisting of hydrogen, —P(O)(OR¹⁴)OR¹⁵, cyano, acyl, aroyl, aryl, alkyl, heteroaryl, heterocycloalkyl, carboxy, carboxyalkyl, optionally substituted alkylthio, optionally substituted arylthio, and a group of structural Formula II 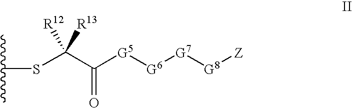
R¹⁴ and R¹⁵ are independently selected from the group consisting of hydrogen, alkyl, aryl, and heteroaryl;
R¹² and R¹³ are independently selected from the group consisting of hydrogen, methyl, and ethyl;
G⁵ are independently selected from the group consisting of a bond, alkenyl, alkoxy, alkoxyalkyl, alkyl, alkylamino, alkylcarbonyl, alkylcarbonylalkyl, alkylcarbonylamino, alkylcarbonylaminoalkyl, alkynyl, amino, aminoalkyl, carbonylalkyl, and carbonylaminoalkyl;
G⁶ are independently selected from the group consisting of optionally substuteted monocyclic heteroaryl, and optionally substuteted polycyclic heteroaryl;
G⁷ is selected from the group consisting of —X³ SO₂N(R⁸)— and —X³N(R⁸)SO₂—;
X³ is selected from the group consisting of a bond or an alkyl of length C₁ to C₃, any carbon atom of which may be optionally substituted;
R⁸ is selected from the group consisting of hydrogen, alkenyl, and alkyl, or alternatively, R⁸ may be joined to G⁵ to form a heterocyclo or heteroaryl ring;
G⁸ is selected from the group consisting of bicyclic aryl, bicyclic heteroaryl, cycloalkyl-fused monocyclic aryl, cycloalkyl-fused monocyclic heteroaryl, heterocycloalkyl-fused monocyclic aryl, and heterocycloalkyl-fused monocyclic heteroaryl, wherein each may be optionally substituted;
Z is selected from the group consisting of null and —U³X⁴U⁴;
U³ is selected from the group consisting of a bond, heterocycloalkyl, —NR¹¹—, —O—, —S—, —C(O)N(R¹¹)—, —N(R¹¹)C(O)—, —S(O)₂N(R¹¹)—, and —N(R¹¹)S(O)—;
R¹¹ is selected from the group consisting of hydrogen, alkenyl, and alkyl;
U⁴ is selected from the group consisting of hydrogen, lower alkyl, lower alkenyl, lower alkynyl, lower alkoxy, lower alkoxyalkyl, lower hydroxyalkyl, aryl, arylalkyl, arylalkenyl, arylalkynyl, heteroaryl, heteroarylalkyl, heteroarylalkenyl, lower cycloalkyl, lower cycloalkylalkyl, heterocycloalkyl, and amino, any of which may be optionally substituted; and
X⁴ is selected from the group consisting of a bond or an alkyl of length C₁ to C₇, any carbon atom of which may be optionally substituted.

33. A method of treatment of an HDAC-related disease comprising the administration of a therapeutically effective amount of said compound as recited in claim 32 to patient in need thereof.

34. A method of treatment of a HDAC-related disease in a patient in need thereof comprising the administration of the following in any order:
i. a therapeutically effective amount of a compound as recited in claim 32; and
ii. together with another chemotherapeutic agent.

35. The method as recited in claim 34 wherein said chemotherapeutic agent is one selected from the group consisting of aromatase inhibitors, antiestrogen, anti-androgen, a gonadorelin agonists, topoisomerase 1 inhibitors, topoisomerase 2 inhibitors, microtubule active agents, alkylating agents, anthracyclines, corticosteroids, IMiDs, protease inhibitors, IGF-1 inhibitors, CD40 antibodies, Smac mimetics, FGF3 modulators, mTOR inhibitors, HDAC inhibitors, IKK inhibitors, P38MAPK inhibitors, HSP90 inhibitors, akt inhibitors, antineoplastic agents, antimetabolites, platin containing compounds, lipid- or protein kinase-targeting agents, protein- or lipid phosphatase-targeting agents, anti-angiogentic agents, agents that induce cell differentiation, bradykinin 1 receptor antagonists, angiotensin II antagonists, cyclooxygenase inhibitors, heparanase inhibitors, lymphokine inhibitors, cytokine inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, and aminopeptidase inhibitors.

36. The method as recited in claim 35 wherein said chemotherapeutic agent is useful for the treatment of multiple myeloma and is selected from the group consisting of alkylating agents, anthracyclines, corticosteroids, IMiDs, protease inhibitors, IGF-1 inhibitors, CD40 antibodies, Smac mimetics, FGF3 modulators, mTOR inhibitors, HDAC inhibitors, IKK inhibitors, P38MAPK inhibitors, HSP90 inhibitors, and akt inhibitors.

37. The method as recited in claim 36, wherein said chemotherapeutic agent is selected from the group consisting of melphalan, doxorubicin, lyophilized, dexamethasone, prednisone, thalidomide, lenalidomide, bortezomib, and NP10052, telomestatin, CHIR258, Rad 001, SAHA, Tubacin, and Perifosine.

38. The method of either claim 33 or claim 34, wherein said disease is a hyperproliferative condition of the human or animal body.

39. The method as recited in claim 38, wherein said hyperproliferative condition is selected from the group consisting of hematologic and nonhematologic cancers.

40. The method as recited in claim 39, wherein said hematologic cancer is selected from the group consisting of multiple myeloma, leukemia, and lymphoma.

41. The method as recited in claim 40, wherein said leukemia is selected from the group consisting of acute and chronic leukemias.

42. The method as recited in claim 41, wherein said acute leukemia is selected from the group consisting of acute lymphocytic leukemia (ALL) and acute nonlymphocytic leukemia (ANLL).

43. The method as recited in claim 41, wherein said chronic leukemia is selected from the group consisting of chronic lymphocytic leukemia (CLL) and chronic myelogenous leukemia (CML).

44. The method as recited in claim 40 wherein said lymphoma is selected from the group consisting of Hodgkin's lymphoma and non-Hodgkin's lymphoma.

45. The method as recited in claim 39 wherein said hematologic cancer is multiple myeloma.

46. The method as recited in claim 39 wherein said hematologic cancer is of low, intermediate, or high grade.

47. The method as recited in claim 39 wherein said nonhematologic cancer is selected from the group consisting of: brain cancer, cancers of the head and neck, lung cancer, breast cancer, cancers of the reproductive system, cancers of the digestive system, pancreatic cancer, and cancers of the urinary system.

48. The method as recited in claim 47 wherein said cancer of the digestive system is a cancer of the upper digestive tract or colorectal cancer.

49. The method as recited in claim 47 wherein said cancer of the urinary system is bladder cancer or renal cell carcinoma.

50. The method as recited in claim 47 wherein said cancer of the reproductive system is prostate cancer.

51. The method as recited in either claim 33 or claim 34, wherein said disease is a hematologic disorder.

52. The method as recited in claim 51, wherein said hematologic disorder is selected from the group consisting of sickle cell anemia, myelodysplastic disorders (MDS), and myeloproliferative disorders.

53. The method as recited in claim 52, wherein said myeloproliferative disorder is selected from the group consisting of polycythemia vera, myelofibrosis and essential thrombocythemia.

54. The method as recited in either claim 33 or claim 34, wherein said disease is a neurological disorder.

55. The method as recited in claim 54, wherein said neurological disorder is selected from the group consisting of epilepsy, neuropathic pain, depression and bipolar disorders.

56. The method as recited in either claim 33 or claim 34, wherein said disease is a cardiovascular condition.

57. The method as recited in claim 56, wherein said cardiovascular condition is selected from the group consisting of cardiac hypertrophy, idiopathic cardiomyopathies, and heart failure.

58. The method as recited in either claim 33 or claim 34 wherein said disease is an autoimmune disease.

59. The method as recited in claim 58, wherein said autoimmune disease is selected from the group consisting of systemic lupus erythromatosus (SLE), multiple sclerosis (MS), and systemic lupus nephritis.

60. The method as recited in either claim 33 or claim 34, wherein said disease is a dermatologic disorder.

61. The method as recited in claim 60, wherein said dermatologic disorder is selected from the group consisting of psoriasis, melanoma, basal cell carcinoma, squamous cell carcinoma, and other non-epithelial skin cancers.

62. The method as recited in either claim 33 or claim 34, wherein said disease is an ophthalmologic disorder.

63. The method as recited in claim 62, wherein said ophthalmologic disorder is selected from the group consisting of dry eye, closed angle glaucoma and wide angle glaucoma.

64. The compound as recited in claim 1 for use in the manufacture of a medicament for the prevention or treatment of a disease or condition ameliorated by the modulation of histone deacetylase (HDAC).
